SEQUENCE LISTING (11 sequences)

SEQ ID NO: 1 - protein, 5 aa, Artificial Sequence
  Anti-IL-12/Il-23p40 antibody complementarity determining region heavy chain 2
TYWLG

SEQ ID NO: 2 - protein, 17 aa, Artificial Seaquence
  Anti-IL-12/IL-23p40 antibody complementarity determining region heavy chain 2
IMSPVDSDIRYSPSFQG

SEQ ID NO: 3 - protein, 10 aa, Artificial sequence
  Anti-IL-12/IL-23p40 antibody complementarity determining region light chain 1
RRPGQGYFDF

SEQ ID NO: 4 - protein, 11 aa, Artificial Sequence
  Anti-IL-12/IL23p40 antibody complementarity determining region light chain 1
RASQGISSWLA

SEQ ID NO: 5 - protein, 7 aa, Artificial Sequence
  Anti-IL-12/IL-23p40 antibody complementarity determining region light chain 2
AASSLQS

SEQ ID NO: 6 - protein, 9 aa, Artificial Sequence
  Anti-IL-12/IL-23p40 Antibody Complementarity Determining Region light chain 3
QQYNIYPYT

SEQ ID NO: 7 - protein, 119 aa, Artificial Sequence
  Anti-IL12-IL23p40 antibody variable heavy chain region
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 8 - protein, 108 aa, Artificial Sequence
  Anti-IL-12/IL23p40 Antibody Varialbe Light Chain Region
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKR

SEQ ID NO: 9 - protein, 503 aa, Artificial Sequence
  Human IL-12 with alpha and beta subunits
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 10 - protein, 449 aa, Artificial Sequence
  Anti-IL-12/IL23p40 Antibody Heavy Chain
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 214 aa, Artificial Sequence
  Anti-IL-12/IL-23p40 Antibody Light Chain
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0112282 A1
(43) Pub. Date: Apr. 14, 2022

(54) Method for Treating Crohn's Disease with Anti-IL12/IL23 Antibody

(71) Applicant: Janssen Biotech, Inc., Horsham, PA (US)

(72) Inventors: Frederic Lavie, Montrouge (FR); Manuela Le Bars, Issy-les-Moulineaux (FR); Michael Plotnick, Floham Park, NJ (US); Sheldon Sloan, Newtown, PA (US)

(21) Appl. No.: 17/496,057

(22) Filed: Oct. 7, 2021

Related U.S. Application Data
(60) Provisional application No. 63/235,188, filed on Aug. 20, 2021.
(60) Provisional application No. 63/089,786, filed on Oct. 9, 2020.

(51) Int. Cl. C07K 16/24; A61K 45/06; A61K 47/18; A61K 47/26; A61P 37/02

(52) U.S. Cl. CPC C07K 16/244; A61K 45/06; A61P 37/02; A61K 47/26; A61K 47/183

(57) ABSTRACT

Methods and compositions for clinical proven safe and effective treatment of Crohn's disease, particularly moderately to severely active Crohn's disease in patients, comprise intravenous initial dosing and subcutaneous maintenance dosing of an anti-IL12/IL23p40 antibody with maintenance dose intervals determined by evaluating clinical indicators.

### CROSS-REFERENCE TO RELATED APPLICATIONS

This application claims priority to U.S. Provisional Application Ser. No. 63/089,786, filed 9 Oct. 2020 and U.S. Provisional Application Ser. No. 63/235,188, filed 20 Aug. 2021. The entire contents of each of the aforementioned application is incorporated herein by reference in their entireties.

### REFERENCE TO SEQUENCE LISTING SUBMITTED ELECTRONICALLY

This application contains a sequence listing, which is submitted electronically via EFS-Web as an ASCII formatted sequence listing with a file name “JBI6409USNP1SEQLIST.txt”, creation date of Oct. 1, 2021 and having a size of 15 KB. The sequence listing submitted via EFS-Web is part of the specification and is herein incorporated by reference in its entirety.

### FIELD OF THE INVENTION

The invention relates to methods of providing a clinically proven safe and clinically proven effective treatment of Crohn's disease, particularly moderately to severely active Crohn's disease in patients whose clinical response is measured at 16 weeks and therapy regimen is evaluated and optionally adjusted with use of intravenous and/or subcutaneous administration of an anti-IL12/23 antibody.

### BACKGROUND OF THE INVENTION

Inflammatory bowel diseases (IBDs), including Crohn's disease (CD), are chronic relapsing disorders characterized by destructive inflammation and epithelial injury in the gastrointestinal (GI) tract (Baumgart and Sandborn, J Clin Invest. 98:1010-1020 (1996); Danese and Fiocchi, N Engl J Med. 365:1715-1725 (2011)).

The involvement of the IL12/23 pathway in the pathogenesis of IBD is well established, and an important role for IL12/IL-23 pathway in intestinal inflammation has been elucidated in colitis (Ahern et al., Immunity. 33(2):279-288 (2010); Uhlig et al., Immunity. 25:309 318 (2006); Yen et al., J Clin Invest. 116(5):1310-1316 (2006)). Early studies showed that treatment with anti-IFNγ (Berg et al., J Clin Invest. 98:1010-1020 (1996); Davidson et al., J Immunol. 161:3143-3149 (1998)) or anti-IL-12p40 monoclonal antibodies (mAb) prevented disease in experimental colitis models, suggesting an important role for type 1 T helper (Th-1) cells in promoting intestinal inflammation (Neurath et al., J Exp Med. 182(5):1281-1290 (1995)).

Currently, there are three classes of biologic agents approved for the treatment of moderately to severely active Crohn's disease: tumor necrosis factor (TNF) antagonist therapies (infliximab, adalimumab, certolizumab), integrin inhibitors (natalizumab and vedolizumab), and an IL12/23 inhibitor (ustekinumab). The efficacy and safety of intravenous (IV) ustekinumab as induction therapy in Crohn's disease have been evaluated in clinical studies CRD3001 and CRD3002. In study CRD3001, subjects with demonstrated prior failure or intolerance to one or more TNF antagonists were evaluated, and in CRD3002 subjects with history of inadequate response to or intolerance of corticosteroids or immunomodulators, but without a history of an inadequate response or intolerance to TNF antagonists were evaluated. In these studies, two IV doses were evaluated: a 130 mg IV fixed dose (˜2 mg/kg on a mg/kg basis) was chosen for the low-dose group, while body-weight range based doses approximating ˜6 mg/kg IV (weight ≤55 kg: ustekinumab 260 mg; weight >55 and ≤85 kg: ustekinumab 390 mg; weight >85 kg: ustekinumab: 520 mg) were chosen as the high-dose group. In both studies, ustekinumab demonstrated clinically significant efficacy compared with placebo and was well-tolerated with a favorable safety profile.

Although the introduction of biologic agents has significantly improved the clinical management of patients with moderately to severely active Crohn's disease, a sizable proportion of the target patient population is non-responsive or will lose response over time. A review of the available data for approved biologic agents highlighted the unmet need in achieving and maintaining long-term remission, especially among patients who have previously failed biologic treatments. In all-treated patients (i.e., all patients who were randomized at Week 0 of the studies evaluated), the estimated rates of clinical remission at 1 year in the biologic failure or intolerance (BIO-Failure) population is around 20%, and ranges from 20% to 50% in the conventional therapy failure or intolerance (CON-Failure) population.

There is a need in the art for improved methods of treating Crohn's disease in order to achieve improved efficacy for a higher percentage of patients, particularly moderately to severely active Crohn's disease.

### BRIEF SUMMARY OF THE INVENTION

The present application relates to clinically proven safe and clinically proven effective methods and compositions for treatment of moderately to severely active Crohn's disease, by administration of an anti-IL12/IL23p40 antibody (anti-IL12/IL23 antibody) to subjects in an initial intravenous dose, a subcutaneous dose eight (8) weeks after the initial dose, measuring indicators of clinical response (efficacy) 16 weeks after the initial dose, based on evaluation of clinical endpoints, biomarkers and/or clinical response, administering the antibody in a subcutaneous dose 16 weeks after the initial dose and every 4 weeks thereafter, every 8 weeks thereafter or every 12 weeks thereafter.

In one general aspect, the application relates to a clinically proven safe and clinically proven effective method of treating moderately to severely active Crohn's disease in a subject in need thereof, comprising administering to the subject a pharmaceutical composition comprising a safe and effective amount of an anti-IL12/23p40 antibody, wherein the antibody comprises a heavy chain variable region and a light chain variable region, the heavy chain variable region comprising: a complementarity determining region heavy chain 1 (CDRH1) amino acid sequence of SEQ ID NO:1; a CDRH2 amino acid sequence of SEQ ID NO:2; and a CDRH3 amino acid sequence of SEQ ID NO:3; and the light chain variable region comprising: a complementarity determining region light chain 1 (CDRL1) amino acid sequence of SEQ ID NO:4; a CDRL2 amino acid sequence of SEQ ID NO:5; and a CDRL3 amino acid sequence of SEQ ID NO:6.

In certain embodiments, methods of the present application comprise intravenously (IV) and/or subcutaneously (SC) administering to the subject a pharmaceutical composition comprising an anti-IL12/23p40 antibody or antigen binding fragment comprising: (i) a heavy chain variable domain amino acid sequence of SEQ ID NO:7; and (ii) a light chain variable domain amino acid sequence of SEQ ID NO:8.

In certain embodiments, methods of the present application comprise intravenously (IV) and/or subcutaneously (SC) administering to the subject a pharmaceutical composition comprising the anti-IL12/23p40 antibody ustekinumab, which comprises: (i) a heavy chain amino acid sequence of SEQ ID NO:10; and (ii) a light chain amino acid sequence of SEQ ID NO:11.

In certain embodiments, the IV dose at week 0 is about 6.0 mg/kg. For example, the IV dose is 260 mg for subjects with body weight ≥35 kg and ≤55 kg, 390 mg for subjects with body weight >55 kg and ≤85 kg, and 520 mg for subjects with body weight >85 kg.

In certain embodiments, the subject treated by methods according to embodiments of the application has had an inadequate response to or are intolerant of a conventional or existing therapy. In some embodiments, the subject had previously failed or were intolerant of a biologic therapy, such as an anti-TNF and/or vedolizumab. In some embodiments, the subject had previously failed or were intolerant of a non-biologic therapy, such as a treatment with corticosteroids, azathioprine (AZA), and/or 6 mercaptopurine (6 MP). In some embodiments, the subject had demonstrated corticosteroid dependence.

In other embodiments, the present invention provides a clinically proven safe and clinically proven effective method of treating moderately to severely active Crohn's disease in a subject, wherein the subject is a responder to the treatment with the antibody and is identified as having a statistically significant improvement in disease activity as determined by clinical indicators and clinical endpoints selected from:
(i) Endoscopic response (decrease from baseline in SES-CD score of ≥50%)
(ii) Overall Endoscopic remission (SES-CD score ≤2)
(iii) Mucosal healing (complete absence of mucosal ulcerations in any ileocolonic segment)
(iv) CDAI 70 response (an improvement of CDAI total score ≥70 points versus baseline)
(v) Clinical response (≥100-point reduction from the baseline CDAI total score, or a CDAI total score <150)
(vi) Clinical remission (CDAI total score of <150 points)
(vii) Change from baseline in biomarkers (fCal and CRP)
(viii) Change from Baseline in the Crohn's Disease Activity Index (CDAI) Score. The CDAI score will be assessed by collecting information on 8 different Crohn's disease-related variables, with scores ranging from 0 to approximately 600. A decrease over time indicates improvement in disease activity.
(ix) Patient-Reported Outcome (PRO)-2 Remission at Week 16 or Week 48 defined based on average daily stool frequency (SF) and average daily abdominal pain (AP) score.
(x) Clinical-Biomarker Response defined using clinical response based on the CDAI score and reduction from baseline in C-reactive protein (CRP) or fecal calprotectin.
(xi) Endoscopic Response measured by the Simple Endoscopic Score for Crohn's Disease (SES-CD). The SES-CD is based on the evaluation of 4 endoscopic components across 5 ileocolonic segments, with a total score ranging from 0 to 56.
(xii) Corticosteroid-Free Clinical Remission at Week 48 defined as CDAI score <150 at Week 48 and not receiving corticosteroids at Week 48.
(xiii) PRO-2 remission at Week 48 defined based on average daily stool frequency (SF) and average daily abdominal pain (AP) score. Fatigue response at Week 12 based on the Patient-Reported Outcomes Measurement Information System (PROWS). Fatigue Short Form 7a contains 7 items that evaluate the severity of fatigue, with higher scores indicating greater fatigue.

In other embodiments, a maintenance dose of the anti-IL12/23p40 antibody is administered every 4 weeks after the treatment at week 16, every 8 weeks after the treatment at week 16 or every 12 weeks after the treatment at week 16 and clinical response is maintained by the subject for at least 48 weeks.

In certain embodiments, the present application provides for a method of treating Crohn's disease in a subject, wherein an anti-IL12/23p40 antibody for use with IV administration is in a pharmaceutical composition comprising a solution comprising 10 mM L-histidine, 8.5% (w/v) sucrose, 0.04% (w/v) polysorbate 80, 0.4 mg/mL L methionine, and 20 μg/mL EDTA disodium salt, dehydrate, at pH 6.0.

In certain embodiments, the present application provides for a clinically proven safe and clinically proven effective method of treating Crohn's disease in a subject, wherein an anti-IL12/IL23p40 antibody for use with subcutaneous administration is in a pharmaceutical composition comprising a solution comprising 6.7 mM L-histidine, 7.6% (w/v) sucrose, 0.004% (w/v) polysorbate 80, at pH 6.0.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

FIG. 1 shows a schematic of the STARDUST treat to target Phase 3b study design of anti-IL-12/IL-23p40 antibody (ustekinumab) in Crohn's disease.

FIG. 2 shows a flow chat of treatment decisions to determine dose adjustment of anti-IL-12/IL-23p40 antibody (ustekinumab) for the trial.

FIG. 3 shows the patient disposition (RAS) in the trial.

FIG. 4 shows the patient disposition (RAS) in the trial of the randomized treat to target (T2T) arm and the standard of care (SoC) arm.

FIG. 5A (NRI) and FIG. 5B (LOCF) show the endoscopic outcomes at Week 48 (left bar T2T and right bar SoC) with p-values (nominal) are based on the Cochran-Mantel-Haenszel test, 2-sided α level of 0.05, stratified by baseline SES-CD score (≤16, >16) and prior exposure to biologics (none or 1). Patients with missing endoscopy assessment are considered as non-responder/no-remitter. Endoscopic response defined as showing a reduction from baseline in SES-CD of ≥25% or 100%. Endoscopic remission defined as a SES-CD score ≤2. Mucosal healing defined as the complete absence of mucosal ulcerations in any ileocolonic segment. Corticosteroid-free endoscopic response is defined as a reduction from baseline in SES-CD score of ≥50% and not taking any corticosteroids for at least 30 days prior to endpoint. LOCF (FIG. 5B)=last observation carried forward; NRI (FIG. 5A)=non-responder imputation.

FIG. 6A (NRI) and FIG. 6B (LOCF) show the clinical outcomes at Week 48 (left bar T2T and right bar SoC) with p-values (nominal) are based on the Cochran-Mantel-Haenszel test, 2-sided α level of 0.05, stratified by baseline SES-CD score (≤16, >16) and prior exposure to biologics (none or 1). Patients with missing data are considered as non-responder/no-remitter. CDAI 70 response defined as showing an improvement of CDAI total score ≥70 points versus baseline. Clinical response defined as ≥100-point reduction from the baseline CDAI total score, or a CDAI total score <150. Clinical remission defined as a CDAI total score of <150 points. At Week 48, high rates of clinical response were achieved in the T2T and SoC arms: 68.2% vs 77.8% (p=0.0212; NRI)/89.5% vs 89.6% (non-significant [NS]; LOCF); clinical remission 61.4% vs 69.7% (NS; NRI)/76.8% vs 78.3% (NS; LOCF). T2T (n=220) and SoC (n=221).

FIG. 7 shows the endoscopic response (SES-CD improvement ≥50% [95% CI] at Week 48 (RAS) with n=220 for T2T (left bar) and n=221 for SoC (right bar). p<0.05. Subjects with missing data were analysed as non-responders. p values (nominal) are based on the CMH test, 2-sided α level of 0.05, stratified by baseline SES-CD score (≤16, >16) and prior exposure to biologics (0 or 1). Subjects who have a missing SES-CD score at Week 48 or who stopped treatment before reaching Week 48 will have their last SES-CD score carried forward. All randomized patients excluding subjects who stopped treatment before reaching Week 48 due to reasons other than lack/loss of efficacy.

FIG. 8 shows the clinical outcomes at Week 48 (RAS NRI) with n=220 for T2T (left bar) and n=221 for SoC (right bar). p<0.05, p-values (nominal) are based on the CMH test, 2-sided α level of 0.05, stratified by baseline SES-CD score (≤16, >16) and prior exposure to biologics (none or 1). Subjects with missing data are considered as non-responder/no-remitter. CDAI 70 response defined as showing an improvement of CDAI total score ≥70 points versus baseline. Clinical response defined as ≥100-point reduction from the baseline CDAI total score, or a CDAI total score <150. Clinical remission defined as a CDAI total score of <150 points. Corticosteroid-free clinical remission at endpoint defined as a CDAI score <150 and not taking any corticosteroids for ≥30 days prior to endpoint assessment.

FIG. 9 shows the clinical outcomes at Week 48 (RAS LOCF) with n=220 for T2T (left bar) and n=221 for SoC (right bar). p-values (nominal) are based on the CMH test, 2-sided α level of 0.05, stratified by baseline SES-CD score (≤16, >16) and prior exposure to biologics (none or 1). ^{b} CDAI 70 response defined as showing an improvement of CDAI total score ≥70 points versus baseline. ^{c} Clinical response defined as ≥100-point reduction from the baseline CDAI total score, or a CDAI total score <150. ^{d} Clinical remission defined as a CDAI total score of <150 points. ^{e} Corticosteroid-free clinical remission at endpoint defined as a CDAI score <150 and not taking any corticosteroids for ≥30 days prior to endpoint assessment.

FIG. 10A (RAS NM) and FIG. 10B (RAS LOCF) show biomarker outcomes relative to number of patients (% in Y axis). For FIG. 10A, p<0.05 with p-values (nominal) are based on the CMH test, 2-sided α level of 0.05, stratified by baseline SES-CD score (<=16, >16) and prior exposure to biologics (none or 1). Subjects with missing data are considered no improvement. fCal improvement is defined as showing a reduction from baseline in fCal of >=50%. Subjects with normalized fCal (<=250 ug/g) at baseline are excluded. CRP improvement is defined as showing a reduction from baseline in CRP of >=50%. Subjects with normalized CRP (<=3 mg/L) at baseline are excluded. Normalized fCal defined as fCal <=250 ug/g. Subjects with Normalized fCal at baseline are excluded. Normalized CRP defined as CRP <=3 mg/L. Subjects with normalized CRP at baseline are excluded. Complete biomarker response is defined as both CRP and fCal normalized. Subjects with normalized CRP and fCal at baseline are excluded as well as subjects with both missing CRP and fCal at baseline are excluded. Normalized CRP defined as CRP <=3 mg/L. Normalized fCal defined as fCal <=250 ug/g. For FIG. 10B, p-values (nominal) are based on the CMH test, 2-sided α level of 0.05, stratified by baseline SES-CD score (<=16, >16) and prior exposure to biologics (none or 1). fCal improvement is defined as showing a reduction from baseline in fCal of >=50%. Subjects with normalized fCal (<=250 ug/g) at baseline are excluded. CRP improvement is defined as showing a reduction from baseline in CRP of >=50%. Subjects with normalized CRP (<=3 mg/L) at baseline are excluded. Normalized fCal defined as fCal <=250 ug/g. Subjects with Normalized fCal at baseline are excluded. Normalized CRP defined as CRP <=3 mg/L. Subjects with normalized CRP at baseline are excluded. Complete biomarker response is defined as both CRP and fCal normalized. Subjects with normalized CRP and fCal at baseline are excluded as well as subjects with both missing CRP and fCal at baseline are excluded. Normalized CRP defined as CRP <=3 mg/L. Normalized fCal defined as fCal <=250 ug/g.

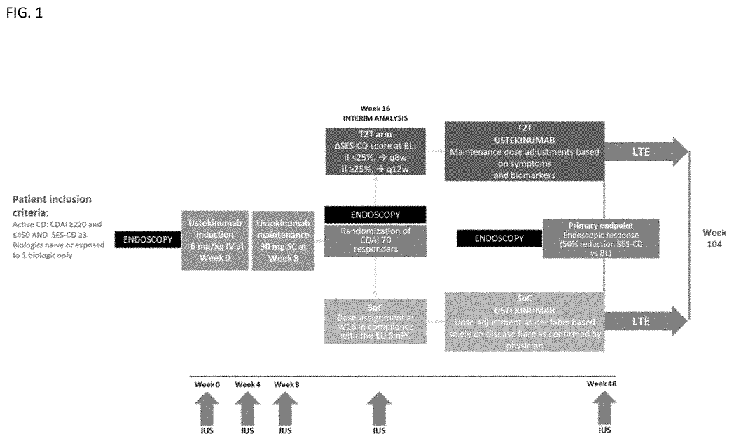

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, “subject” means any animal, preferably a mammal, most preferably a human, whom will be or has been treated by a method according to an embodiment of the invention. The term “mammal” as used herein, encompasses any mammal. Examples of mammals include, but are not limited to, cows, horses, sheep, pigs, cats, dogs, mice, rats, rabbits, guinea pigs, non-human primates (NHPs) such as monkeys or apes, humans, etc., more preferably a human.

As used herein, the term “in combination”, in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

As used herein, an “anti-IL12/23p40 antibody,” or “IL12/23 antibody,” refers to a monoclonal antibody (mAb) or antigen binding fragment thereof, that binds the 40 kDa (p40) subunit shared by the cytokines interleukin-12 and interleukin-23 (IL12/23p40). The antibody can affect at least one of IL12/23 activity or function, such as but not limited to, RNA, DNA or protein synthesis, IL12/23 release, IL12/23 receptor signaling, membrane IL12/23 cleavage, IL12/23 activity, IL12/23 production and/or synthesis.

The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments that bind to a mammalian IL-12/23. For example, antibody fragments capable of binding to IL-12/23 or portions thereof, including, but not limited to, Fab (e.g., by papain digestion), Fab′ (e.g., by pepsin digestion and partial reduction) and F(ab′)2 (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the invention (see, e.g., Colligan, Immunology, supra).

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)₂ heavy chain portion can be designed to include DNA sequences encoding the C_{H}1 domain and/or hinge region of the heavy chain. The various portions of antibodies can be joined together chemically by conventional techniques, or can be prepared as a contiguous protein using genetic engineering techniques.

As used herein, the term “human antibody” refers to an antibody in which substantially every part of the protein (e.g., CDR, framework, C_{L}, C_{H} domains (e.g., C_{H}1, C_{H}2, C_{H}3), hinge, (V_{L}, V_{H})) is substantially non-immunogenic in humans, with only minor sequence changes or variations. A “human antibody” can also be an antibody that is derived from or closely matches human germline immunoglobulin sequences. Human antibodies can include amino acid residues not encoded by germline immunoglobulin sequences (e.g., mutations introduced by random or site-specific mutagenesis in vitro or by somatic mutation in vivo). Often, this means that the human antibody is substantially non-immunogenic in humans. Human antibodies have been classified into groupings based on their amino acid sequence similarities. Accordingly, using a sequence similarity search, an antibody with a similar linear sequence can be chosen as a template to create a human antibody. Similarly, antibodies designated primate (monkey, baboon, chimpanzee, etc.), rodent (mouse, rat, rabbit, guinea pig, hamster, and the like) and other mammals designate such species, sub-genus, genus, sub-family, and family specific antibodies. Further, chimeric antibodies can include any combination of the above. Such changes or variations optionally and preferably retain or reduce the immunogenicity in humans or other species relative to non-modified antibodies. Thus, a human antibody is distinct from a chimeric or humanized antibody.

It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

Anti-IL12/23p40 antibodies (also termed IL12/23p40 antibodies) useful in the methods and compositions of the present invention can optionally be characterized by high affinity binding to IL12/23p40, optionally and preferably, having low toxicity. In particular, an antibody, specified fragment or variant of the invention, where the individual components, such as the variable region, constant region and framework, individually and/or collectively, optionally and preferably possess low immunogenicity, is useful in the present invention. The antibodies that can be used in the invention are optionally characterized by their ability to treat subjects for extended periods with measurable alleviation of symptoms and low and/or acceptable toxicity. Low or acceptable immunogenicity and/or high affinity, as well as other suitable properties, can contribute to the therapeutic results achieved. “Low immunogenicity” is defined herein as raising significant HAHA, HACA or HAMA responses in less than about 75%, or preferably less than about 50% of the subjects treated and/or raising low titres in the subject treated (less than about 300, preferably less than about 100 measured with a double antigen enzyme immunoassay) (Elliott et al., Lancet 344:1125-1127 (1994), entirely incorporated herein by reference). “Low immunogenicity” can also be defined as the incidence of titrable levels of antibodies to the anti-IL-12 antibody in subjects treated with anti-IL-12 antibody as occurring in less than 25% of subjects treated, preferably, in less than 10% of subjects treated with the recommended dose for the recommended course of therapy during the treatment period.

The terms “clinically proven efficacy” and “clinically proven effective” as used herein in the context of a dose, dosage regimen, treatment or method refer to the effectiveness of a particular dose, dosage or treatment regimen. Efficacy can be measured based on change in the course of the disease in response to an agent of the present invention. For example, an anti-IL12/23p40 of the present invention (e.g., ustekinumab) is administered to a subject in an amount and for a time sufficient to induce an improvement, preferably a sustained improvement, in at least one indicator that reflects the severity of the disorder that is being treated. Various indicators that reflect the extent of the subject's illness, disease or condition can be assessed for determining whether the amount and time of the treatment is sufficient. Such indicators include, for example, clinically recognized indicators of disease severity, symptoms, or manifestations of the disorder in question. The degree of improvement generally is determined by a physician, who can make this determination based on signs, symptoms, biopsies, or other test results, and who can also employ questionnaires that are administered to the subject, such as quality-of-life questionnaires developed for a given disease. For example, an anti-IL12/23p40 or anti-IL23 antibody of the present invention can be administered to achieve clinical remission or an improvement in a subject's condition related to Crohn's disease.

Improvement can be indicated by an improvement in an index of disease activity, by amelioration of clinical symptoms or by any other measure of disease activity as described herein. Once such index of disease is the Crohn's disease activity index (CDAI) score or Simple Endoscopic Score in CD (SES-CD).

The term “clinical response” as used herein as it relates to a subject's response to drug administration, and can also refer to “clinic remission” as measured in Crohn's disease and known in the art.

The term “clinically proven safe,” as it relates to a dose, dosage regimen, treatment or method with anti-IL12/IL-23p40 antibody of the present invention (e.g., ustekinumab), refers to a favorable risk:benefit ratio with an acceptable frequency and/or acceptable severity of treatment-emergent adverse events (referred to as AEs or TEAEs) compared to the standard of care or to another comparator. As used herein, “adverse event,” “treatment-emergent adverse event,” and “adverse reaction” mean any harm, unfavorable, unintended or undesired sign or outcome associated with or caused by administration of a pharmaceutical composition or therapeutic. It is an untoward medical occurrence in a subject administered a medicinal product. However, abnormal values or observations are not reported as adverse events unless considered clinically significant by the investigator. As used herein, when referring to an adverse event, “clinically apparent” means clinically significant as determined by a medical doctor or an investigator using standard acceptable to those of ordinary skill in the art. When the harm or undesired outcome of adverse events reaches such a level of severity, a regulatory agency can deem the pharmaceutical composition or therapeutic unacceptable for the proposed use. In particular, “safe” as it relates to a dose, dosage regimen or treatment with an anti-IL12/23p40 antibody of the present invention refers to with an acceptable frequency and/or acceptable severity of adverse events associated with administration of the antibody if attribution is considered to be possible, probable, or very likely due to the use of the anti-IL12/23p40 antibody.

As used herein, unless otherwise noted, the term “clinically proven” (used independently or to modify the terms “safe” and/or “effective”) shall mean that it has been proven by a clinical trial wherein the clinical trial has met the approval standards of U.S. Food and Drug Administration, EMEA or a corresponding national regulatory agency. For example, the clinical study may be an adequately sized, randomized, double-blinded study used to clinically prove the effects of the drug.

As used herein, a dosage amount of an anti-IL12/IL23p40 antibody in “mg/kg” refers to the amount of the anti-IL12/IL23p40 antibody in milligrams per kilogram of the body weight of a subject to be administered with the antibody.

Antibodies of the Present Invention—Production and Generation

At least one anti-IL12/23p40 used in the method of the present invention can be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, NY (1987-2001); Sambrook, et al., Molecular Cloning: A Laboratory Manual, 2nd Edition, Cold Spring Harbor, N.Y. (1989); Harlow and Lane, antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, NY, (1997-2001), each entirely incorporated herein by reference.

Human antibodies that are specific for human IL-12/23p40 proteins or fragments thereof can be raised against an appropriate immunogenic antigen, such as an isolated IL-12/23p40 protein, IL-23 protein and/or a portion thereof (including synthetic molecules, such as synthetic peptides). Other specific or general mammalian antibodies can be similarly raised. Preparation of immunogenic antigens, and monoclonal antibody production can be performed using any suitable technique in view of the present disclosure.

In one approach, a hybridoma is produced by fusing a suitable immortal cell line (e.g., a myeloma cell line, such as, but not limited to, Sp2/0, Sp2/0-AG14, NSO, NS1, NS2, AE-1, L.5, L243, P3X63Ag8.653, Sp2 SA3, Sp2 MAI, Sp2 SS1, Sp2 SA5, U937, MLA 144, ACT IV, MOLT4, DA-1, JURKAT, WEHI, K-562, COS, RAJI, NIH 3T3, HL-60, MLA 144, NAMALWA, NEURO 2A, or the like, or heteromylomas, fusion products thereof, or any cell or fusion cell derived therefrom, or any other suitable cell line as known in the art) (see, e.g., www.atcc.org, www.lifetech.com, and the like), with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. See, e.g., Ausubel, supra, and Colligan, Immunology, supra, chapter 2, entirely incorporated herein by reference.

Antibody producing cells can also be obtained from the peripheral blood or, preferably, the spleen or lymph nodes, of humans or other suitable animals that have been immunized with the antigen of interest. Any other suitable host cell can also be used for expressing heterologous or endogenous nucleic acid encoding an antibody, specified fragment or variant thereof, of the present invention. The fused cells (hybridomas) or recombinant cells can be isolated using selective culture conditions or other suitable known methods, and cloned by limiting dilution or cell sorting, or other known methods. Cells which produce antibodies with the desired specificity can be selected by a suitable assay (e.g., ELISA).

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from Cambridge antibody Technologies, Cambridgeshire, UK; MorphoSys, Martinsreid/Planegg, DE; Biovation, Aberdeen, Scotland, UK; Biolnvent, Lund, Sweden; Dyax Corp., Enzon, Affymax/Biosite; Xoma, Berkeley, Calif.; Ixsys. See, e.g., EP 368,684, PCT/GB91/01134; PCT/GB92/01755; PCT/GB92/002240; PCT/GB92/00883; PCT/GB93/00605; U.S. Ser. No. 08/350,260 (5/12/94); PCT/GB94/01422; PCT/GB94/02662; PCT/GB97/01835; (CAT/MRC); WO90/14443; WO90/14424; WO90/14430; PCT/US94/1234; WO92/18619; WO96/07754; (Scripps); WO96/13583, WO97/08320 (MorphoSys); WO95/16027 (BioInvent); WO88/06630; WO90/3809 (Dyax); U.S. Pat. No. 4,704,692 (Enzon); PCT/US91/02989 (Affymax); WO89/06283; EP 371 998; EP 550 400; (Xoma); EP 229 046; PCT/US91/07149 (Ixsys); or stochastically generated peptides or proteins—U.S. Pat. Nos. 5,723,323, 5,763,192, 5,814,476, 5,817,483, 5,824,514, 5,976,862, WO 86/05803, EP 590 689 (Ixsys, predecessor of Applied Molecular Evolution (AME), each entirely incorporated herein by reference)) or that rely upon immunization of transgenic animals (e.g., SCID mice, Nguyen et al., Microbiol. Immunol. 41:901-907 (1997); Sandhu et al., Crit. Rev. Biotechnol. 16:95-118 (1996); Eren et al., Immunol. 93:154-161 (1998), each entirely incorporated by reference as well as related patents and applications) that are capable of producing a repertoire of human antibodies, as known in the art and/or as described herein. Such techniques, include, but are not limited to, ribosome display (Hanes et al., Proc. Natl. Acad. Sci. USA, 94:4937-4942 (Can 1997); Hanes et al., Proc. Natl. Acad. Sci. USA, 95:14130-14135 (November 1998)); single cell antibody producing technologies (e.g., selected lymphocyte antibody method (“SLAM”) (U.S. Pat. No. 5,627,052, Wen et al., J. Immunol. 17:887-892 (1987); Babcook et al., Proc. Natl. Acad. Sci. USA 93:7843-7848 (1996)); gel microdroplet and flow cytometry (Powell et al., Biotechnol. 8:333-337 (1990); One Cell Systems, Cambridge, Mass.; Gray et al., J. Imm. Meth. 182:155-163 (1995); Kenny et al., Bio/Technol. 13:787-790 (1995)); B-cell selection (Steenbakkers et al., Molec. Biol. Reports 19:125-134 (1994); Jonak et al., Progress Biotech, Vol. 5, In Vitro Immunization in Hybridoma Technology, Borrebaeck, ed., Elsevier Science Publishers B.V., Amsterdam, Netherlands (1988)).

Methods for engineering or humanizing non-human or human antibodies can also be used and are well known in the art. Generally, a humanized or engineered antibody has one or more amino acid residues from a source that is non-human, e.g., but not limited to, mouse, rat, rabbit, non-human primate or other mammal. These non-human amino acid residues are replaced by residues often referred to as “import” residues, which are typically taken from an “import” variable, constant or other domain of a known human sequence.

Known human Ig sequences are disclosed, e.g., www.ncbi.nlm.nih.gov/entrez/query.fcgi; www.ncbi.nih.gov/igblast; www.atcc.org/phage/hdb.html; www.mrc-cpe.cam.ac.uk/ALIGNMENTS.php; www.kabatdatabase.com/top.html; ftp.ncbi.nih.gov/repository/kabat; www.sciquest.com; www.abcam.com; www.antibodyresource.com/onlinecomp.html; www.public.iastate.edu/˜pedro/research tools.html; www.whfreeman.com/immunology/CH05/kuby05.htm; www.hhmi.org/grants/lectures/1996/vlab; www.path.cam.ac.uk/˜mrc7/mikeimages.html; mcb.harvard.edu/BioLinks/Immunology.html; www.immunologylink.com; pathbox.wustl.edu/˜hcenter/index.html; www.appliedbiosystems.com; www.nal.usda.gov/awic/pubs/antibody; www.m.ehime-u.ac.jp/˜yasuhito/Elisa.html; www.biodesign.com; www.cancerresearchuk.org; www.biotech.ufl.edu; www.isac-net.org; baserv.uci.kun.nl/˜jraats/links1.html; www.recab.uni-hd.de/immuno.bme.nwu.edu; www.mrc-cpe.cam.ac.uk; www.ibt.unam.mx/vir/V_mice.html; http://www.bioinforg.uk/abs; antibody.bath.ac.uk; www.unizh.ch; www.cryst.bbk.ac.uk/˜ubcg07s; www.nimr.mrc.ac.uk/CC/ccaewg/ccaewg.html; www.path.cam.ac.uk/˜mrc7/humanisation/TAHHP.html; www.ibt.unam.mx/vir/structure/stat_aim.html; www.biosci.missouri.edu/smithgp/index.html; www.jerini.de; Kabat et al., Sequences of Proteins of Immunological Interest, U.S. Dept. Health (1983), each entirely incorporated herein by reference.

Such imported sequences can be used to reduce immunogenicity or reduce, enhance or modify binding, affinity, on-rate, off-rate, avidity, specificity, half-life, or any other suitable characteristic, as known in the art. In general, the CDR residues are directly and most substantially involved in influencing antigen binding. Accordingly, part or all of the non-human or human CDR sequences are maintained while the non-human sequences of the variable and constant regions can be replaced with human or other amino acids.

Antibodies can also optionally be humanized or human antibodies engineered with retention of high affinity for the antigen and other favorable biological properties. To achieve this goal, humanized (or human) antibodies can be optionally prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, framework (FR) residues can be selected and combined from the consensus and import sequences so that the desired antibody characteristic, such as increased affinity for the target antigen(s), is achieved.

In addition, the human anti-IL12/23p40 antibody used in the method of the present invention can comprise a human germline light chain framework. In particular embodiments, the light chain germline sequence is selected from human VK sequences including, but not limited to, A1, A10, A11, A14, A17, A18, A19, A2, A20, A23, A26, A27, A3, A30, A5, A7, B2, B3, L1, L10, L11, L12, L14, L15, L16, L18, L19, L2, L20, L22, L23, L24, L25, L4/18a, L5, L6, L8, L9, O1, O11, O12, O14, O18, O2, O4, and O8. In certain embodiments, this light chain human germline framework is selected from V1-11, V1-13, V1-16, V1-17, V1-18, V1-19, V1-2, V1-20, V1-22, V1-3, V1-4, V1-5, V1-7, V1-9, V2-1, V2-11, V2-13, V2-14, V2-15, V2-17, V2-19, V2-6, V2-7, V2-8, V3-2, V3-3, V3-4, V4-1, V4-2, V4-3, V4-4, V4-6, V5-1, V5-2, V5-4, and V5-6.

In other embodiments, the human anti-IL-12/23p40 (or anti-IL-23) specific antibody used in the method of the present invention can comprise a human germline heavy chain framework. In particular embodiments, this heavy chain human germline framework is selected from VH1-18, VH1-2, VH1-24, VH1-3, VH1-45, VH1-46, VH1-58, VH1-69, VH1-8, VH2-26, VH2-5, VH2-70, VH3-11, VH3-13, VH3-15, VH3-16, VH3-20, VH3-21, VH3-23, VH3-30, VH3-33, VH3-35, VH3-38, VH3-43, VH3-48, VH3-49, VH3-53, VH3-64, VH3-66, VH3-7, VH3-72, VH3-73, VH3-74, VH3-9, VH4-28, VH4-31, VH4-34, VH4-39, VH4-4, VH4-59, VH4-61, VH5-51, VH6-1, and VH7-81.

In particular embodiments, the light chain variable region and/or heavy chain variable region comprises a framework region or at least a portion of a framework region (e.g., containing 2 or 3 subregions, such as FR2 and FR3). In certain embodiments, at least FRL1, FRL2, FRL3, or FRL4 is fully human. In other embodiments, at least FRH1, FRH2, FRH3, or FRH4 is fully human. In some embodiments, at least FRL1, FRL2, FRL3, or FRL4 is a germline sequence (e.g., human germline) or comprises human consensus sequences for the particular framework (readily available at the sources of known human Ig sequences described above). In other embodiments, at least FRH1, FRH2, FRH3, or FRH4 is a germline sequence (e.g., human germline) or comprises human consensus sequences for the particular framework. In preferred embodiments, the framework region is a fully human framework region.

Humanization or engineering of antibodies of the present invention can be performed using any known method, such as but not limited to those described in, Winter (Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. 5,723,323, 5,976,862, 5,824,514, 5,817,483, 5,814,476, 5,763,192, 5,723,323, 5,766886, 5714352, 6204023, 6180370, 5693762, 5530101, 5585089, 5225539; 4816567, PCT/: US98/16280, US96/18978, US91/09630, US91/05939, US94/01234, GB89/01334, GB91/01134, GB92/01755; WO90/14443, WO90/14424, WO90/14430, EP 229246, each entirely incorporated herein by reference, included references cited therein.

In certain embodiments, the antibody comprises an altered (e.g., mutated) Fc region. For example, in some embodiments, the Fc region has been altered to reduce or enhance the effector functions of the antibody. In some embodiments, the Fc region is an isotype selected from IgM, IgA, IgG, IgE, or other isotype. Alternatively, or additionally, it can be useful to combine amino acid modifications with one or more further amino acid modifications that alter C1q binding and/or the complement dependent cytotoxicity function of the Fc region of an IL-23 binding molecule. The starting polypeptide of particular interest can be one that binds to C1q and displays complement dependent cytotoxicity (CDC). Polypeptides with pre-existing C1q binding activity, optionally further having the ability to mediate CDC can be modified such that one or both of these activities are enhanced. Amino acid modifications that alter C1q and/or modify its complement dependent cytotoxicity function are described, for example, in WO0042072, which is hereby incorporated by reference.

As disclosed above, one can design an Fc region of the human anti-IL12/23p40 antibody of the present invention with altered effector function, e.g., by modifying C1q binding and/or FcγR binding and thereby changing complement dependent cytotoxicity (CDC) activity and/or antibody-dependent cell-mediated cytotoxicity (ADCC) activity. “Effector functions” are responsible for activating or diminishing a biological activity (e.g., in a subject). Examples of effector functions include, but are not limited to: C1q binding; CDC; Fc receptor binding; ADCC; phagocytosis; down regulation of cell surface receptors (e.g., B cell receptor; BCR), etc. Such effector functions can require the Fc region to be combined with a binding domain (e.g., an antibody variable domain) and can be assessed using various assays (e.g., Fc binding assays, ADCC assays, CDC assays, etc.).

For example, one can generate a variant Fc region of the human anti-IL12/23p40 antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities can be increased, and, optionally, also the other activity reduced (e.g., to generate an Fc region variant with improved ADCC activity, but reduced CDC activity and vice versa).

Fc mutations can also be introduced in engineer to alter their interaction with the neonatal Fc receptor (FcRn) and improve their pharmacokinetic properties. A collection of human Fc variants with improved binding to the FcRn have been described (Shields et al., (2001). High resolution mapping of the binding site on human IgG1 for FcγRI, FcγRII, FcγRIII, and FcRn and design of IgG1 variants with improved binding to the FcγR, J. Biol. Chem. 276:6591-6604).

Another type of amino acid substitution serves to alter the glycosylation pattern of the Fc region of the human anti-IL12/23p40 antibody. Glycosylation of an Fc region is typically either N-linked or O-linked. N-linked refers to the attachment of the carbohydrate moiety to the side chain of an asparagine residue. O-linked glycosylation refers to the attachment of one of the sugars N-aceylgalactosamine, galactose, or xylose to a hydroxyamino acid, most commonly serine or threonine, although 5-hydroxyproline or 5-hydroxylysine can also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern can be altered, for example, by deleting one or more glycosylation site(s) found in the polypeptide, and/or adding one or more glycosylation sites that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration can also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the human anti-IL12/23p40 antibody of the present invention is expressed in cells that express beta (1,4)-N-acetylglucosaminyltransferase III (GnT III), such that GnT III adds GlcNAc to the human anti-IL-12/23p40 (or anti-IL-23) antibody. Methods for producing antibodies in such a fashion are provided in WO/9954342, WO/03011878, patent publication 20030003097A1, and Umana et al., Nature Biotechnology, 17:176-180, February 1999; all of which are herein specifically incorporated by reference in their entireties.

The human anti-IL12/23p40 antibody can also be optionally generated by immunization of a transgenic animal (e.g., mouse, rat, hamster, non-human primate, and the like) capable of producing a repertoire of human antibodies, as described herein and/or as known in the art. Cells that produce a human anti-IL12/23p40 antibody can be isolated from such animals and immortalized using suitable methods, such as the methods described herein.

Transgenic mice that can produce a repertoire of human antibodies that bind to human antigens can be produced by known methods (e.g., but not limited to, U.S. Pat. Nos. 5,770,428, 5,569,825, 5,545,806, 5,625,126, 5,625,825, 5,633,425, 5,661,016 and 5,789,650 issued to Lonberg et al.; Jakobovits et al. WO 98/50433, Jakobovits et al. WO 98/24893, Lonberg et al. WO 98/24884, Lonberg et al. WO 97/13852, Lonberg et al. WO 94/25585, Kucherlapate et al. WO 96/34096, Kucherlapate et al. EP 0463 151 B1, Kucherlapate et al. EP 0710 719 A1, Surani et al. U.S. Pat. No. 5,545,807, Bruggemann et al. WO 90/04036, Bruggemann et al. EP 0438 474 B1, Lonberg et al. EP 0814 259 A2, Lonberg et al. GB 2 272 440 A, Lonberg et al. Nature 368:856-859 (1994), Taylor et al., Int. Immunol. 6(4)579-591 (1994), Green et al, Nature Genetics 7:13-21 (1994), Mendez et al., Nature Genetics 15:146-156 (1997), Taylor et al., Nucleic Acids Research 20(23):6287-6295 (1992), Tuaillon et al., Proc Natl Acad Sci USA 90(8)3720-3724 (1993), Lonberg et al., Int Rev Immunol 13(1):65-93 (1995) and Fishwald et al., Nat Biotechnol 14(7):845-851 (1996), which are each entirely incorporated herein by reference). Generally, these mice comprise at least one transgene comprising DNA from at least one human immunoglobulin locus that is functionally rearranged, or which can undergo functional rearrangement. The endogenous immunoglobulin loci in such mice can be disrupted or deleted to eliminate the capacity of the animal to produce antibodies encoded by endogenous genes.

Screening antibodies for specific binding to similar proteins or fragments can be conveniently achieved using peptide display libraries. This method involves the screening of large collections of peptides for individual members having the desired function or structure. Antibody screening of peptide display libraries is well known in the art. The displayed peptide sequences can be from 3 to 5000 or more amino acids in length, frequently from 5-100 amino acids long, and often from about 8 to 25 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT Patent Publication Nos. 91/17271, 91/18980, 91/19818, and 93/08278.

Other systems for generating libraries of peptides have aspects of both in vitro chemical synthesis and recombinant methods. See, PCT Patent Publication Nos. 92/05258, 92/14843, and 96/19256. See also, U.S. Pat. Nos. 5,658,754; and 5,643,768. Peptide display libraries, vector, and screening kits are commercially available from such suppliers as Invitrogen (Carlsbad, Calif.), and Cambridge antibody Technologies (Cambridgeshire, UK). See, e.g., U.S. Pat. Nos. 4,704,692, 4,939,666, 4,946,778, 5,260,203, 5,455,030, 5,518,889, 5,534,621, 5,656,730, 5,763,733, 5,767,260, 5,856,456, assigned to Enzon; U.S. Pat. Nos. 5,223,409, 5,403,484, 5,571,698, 5,837,500, assigned to Dyax, 5427908, 5580717, assigned to Affymax; 5885793, assigned to Cambridge antibody Technologies; 5750373, assigned to Genentech, 5618920, 5595898, 5576195, 5698435, 5693493, 5698417, assigned to Xoma, Colligan, supra; Ausubel, supra; or Sambrook, supra, each of the above patents and publications entirely incorporated herein by reference.

Antibodies used in the method of the present invention can also be prepared using at least one anti-IL12/23p40 antibody encoding nucleic acid to provide transgenic animals or mammals, such as goats, cows, horses, sheep, rabbits, and the like, that produce such antibodies in their milk. Such animals can be provided using known methods. See, e.g., but not limited to, U.S. Pat. Nos. 5,827,690; 5,849,992; 4,873,316; 5,849,992; 5,994,616; 5,565,362; 5,304,489, and the like, each of which is entirely incorporated herein by reference.

Antibodies used in the method of the present invention can additionally be prepared using at least one anti-IL12/23p40 antibody encoding nucleic acid to provide transgenic plants and cultured plant cells (e.g., but not limited to, tobacco and maize) that produce such antibodies, specified portions or variants in the plant parts or in cells cultured therefrom. As a non-limiting example, transgenic tobacco leaves expressing recombinant proteins have been successfully used to provide large amounts of recombinant proteins, e.g., using an inducible promoter. See, e.g., Cramer et al., Curr. Top. Microbol. Immunol. 240:95-118 (1999) and references cited therein. Also, transgenic maize have been used to express mammalian proteins at commercial production levels, with biological activities equivalent to those produced in other recombinant systems or purified from natural sources. See, e.g., Hood et al., Adv. Exp. Med. Biol. 464:127-147 (1999) and references cited therein. Antibodies have also been produced in large amounts from transgenic plant seeds including antibody fragments, such as single chain antibodies (scFv's), including tobacco seeds and potato tubers. See, e.g., Conrad et al., Plant Mol. Biol. 38:101-109 (1998) and references cited therein. Thus, antibodies of the present invention can also be produced using transgenic plants, according to known methods. See also, e.g., Fischer et al., Biotechnol. Appl. Biochem. 30:99-108 (October, 1999), Ma et al., Trends Biotechnol. 13:522-7 (1995); Ma et al., Plant Physiol. 109:341-6 (1995); Whitelam et al., Biochem. Soc. Trans. 22:940-944 (1994); and references cited therein. Each of the above references is entirely incorporated herein by reference.

The antibodies used in the method of the invention can bind human IL12/IL23p40 with a wide range of affinities (KD). In a preferred embodiment, a human mAb can optionally bind human IL12/IL23p40 with high affinity. For example, a human mAb can bind human IL12/23p40 with a KD equal to or less than about 10-7 M, such as but not limited to, 0.1-9.9 (or any range or value therein)×10-7, 10-8, 10-9, 10-10, 10-11, 10-12, 10-13 or any range or value therein.

The affinity or avidity of an antibody for an antigen can be determined experimentally using any suitable method. (See, for example, Berzofsky, et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein). The measured affinity of a particular antibody-antigen interaction can vary if measured under different conditions (e.g., salt concentration, pH). Thus, measurements of affinity and other antigen-binding parameters (e.g., KD, Ka, Kd) are preferably made with standardized solutions of antibody and antigen, and a standardized buffer, such as the buffer described herein.

Vectors and Host Cells

The present invention also relates to vectors that include isolated nucleic acid molecules, host cells that are genetically engineered with the recombinant vectors, and the production of at least one anti-IL12/23p40 antibody by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, methotrexate (MTX), dihydrofolate reductase (DHFR, U.S. Pat. Nos. 4,399,216; 4,634,665; 4,656,134; 4,956,288; 5,149,636; 5,179,017, ampicillin, neomycin (G418), mycophenolic acid, or glutamine synthetase (GS, U.S. Pat. Nos. 5,122,464; 5,770,359; 5,827,739) resistance for eukaryotic cell culture, and tetracycline or ampicillin resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

At least one antibody used in the method of the present invention can be expressed in a modified form, such as a fusion protein, and can include not only secretion signals, but also additional heterologous functional regions. For instance, a region of additional amino acids, particularly charged amino acids, can be added to the N-terminus of an antibody to improve stability and persistence in the host cell, during purification, or during subsequent handling and storage. Also, peptide moieties can be added to an antibody of the present invention to facilitate purification. Such regions can be removed prior to final preparation of an antibody or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid encoding a protein used in the method of the present invention. Alternatively, nucleic acids can be expressed in a host cell by turning on (by manipulation) in a host cell that contains endogenous DNA encoding an antibody. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the antibodies, specified portions or variants thereof, are mammalian cells. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Ag14 cells (ATCC Accession Number CRL-1851). In a particularly preferred embodiment, the recombinant cell is a P3X63Ab8.653 or a SP2/0-Ag14 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human immunoglobulin promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present invention are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

Purification of an Antibody

An anti-IL12/23p40 antibody can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, NY, (1997-2001), e.g., Chapters 1, 4, 6, 8, 9, 10, each entirely incorporated herein by reference.

Antibodies used in the method of the present invention include naturally purified products, products of chemical synthetic procedures, and products produced by recombinant techniques from a eukaryotic host, including, for example, yeast, higher plant, insect and mammalian cells. Depending upon the host employed in a recombinant production procedure, the antibody can be glycosylated or can be non-glycosylated, with glycosylated preferred. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

Anti-IL12/23p40 Antibodies

An anti-IL12/23p40 antibody according to the present invention includes any protein or peptide containing molecule that comprises at least a portion of an immunoglobulin molecule, such as but not limited to, at least one ligand binding portion (LBP), such as but not limited to, a complementarity determining region (CDR) of a heavy or light chain or a ligand binding portion thereof, a heavy chain or light chain variable region, a framework region (e.g., FR1, FR2, FR3, FR4 or fragment thereof, further optionally comprising at least one substitution, insertion or deletion), a heavy chain or light chain constant region, (e.g., comprising at least one CH1, hinge1, hinge2, hinge3, hinge4, CH2, or CH3 or fragment thereof, further optionally comprising at least one substitution, insertion or deletion), or any portion thereof, that can be incorporated into an antibody. An antibody can include or be derived from any mammal, such as but not limited to, a human, a mouse, a rabbit, a rat, a rodent, a primate, or any combination thereof, and the like.

Preferably, the human antibody or antigen-binding fragment binds human IL12/23p40 and, thereby, partially or substantially neutralizes at least one biological activity of the protein. An antibody, or specified portion or variant thereof, that partially or preferably substantially neutralizes at least one biological activity of at least one IL12/23p40 protein or fragment can bind the protein or fragment and thereby inhibit activities mediated through the binding of IL12/23p40 or IL-23 to the IL-12 and/or IL-23 receptor or through other IL12/23p40 or IL-23-dependent or mediated mechanisms. As used herein, the term “neutralizing antibody” refers to an antibody that can inhibit an IL12/23p40 or IL-23-dependent activity by about 20-120%, preferably by at least about 10, 20, 30, 40, 50, 55, 60, 65, 70, 75, 80, 85, 90, 91, 92, 93, 94, 95, 96, 97, 98, 99, 100% or more depending on the assay. The capacity of an anti-IL12/23p40 or IL-23 antibody to inhibit an IL12/23p40 or IL-23-dependent activity is preferably assessed by at least one suitable IL12/23p40 or IL-23 protein or receptor assay, as described herein and/or as known in the art. A human antibody can be of any class (IgG, IgA, IgM, IgE, IgD, etc.) or isotype and can comprise a kappa or lambda light chain. In one embodiment, the human antibody comprises an IgG heavy chain or defined fragment, for example, at least one of isotypes, IgG1, IgG2, IgG3 or IgG4 (e.g., γ1, γ2, γ3, γ4). Antibodies of this type can be prepared by employing a transgenic mouse or other transgenic non-human mammal comprising at least one human light chain (e.g., IgG, IgA, and IgM) transgenes as described herein and/or as known in the art. In another embodiment, the human antibody comprises an IgG1 heavy chain and an IgG1 light chain.

An antibody binds at least one specified epitope specific to at least one IL12/23p40 protein, subunit, fragment, portion or any combination thereof. The at least one epitope can comprise at least one antibody binding region that comprises at least one portion of the protein, which epitope is preferably comprised of at least one extracellular, soluble, hydrophillic, external or cytoplasmic portion of the protein.

Generally, the human antibody or antigen-binding fragment will comprise an antigen-binding region that comprises at least one human complementarity determining region (CDR1, CDR2 and CDR3) or variant of at least one heavy chain variable region and at least one human complementarity determining region (CDR1, CDR2 and CDR3) or variant of at least one light chain variable region. The CDR sequences can be derived from human germline sequences or closely match the germline sequences. For example, the CDRs from a synthetic library derived from the original non-human CDRs can be used. These CDRs can be formed by incorporation of conservative substitutions from the original non-human sequence. In another particular embodiment, the antibody or antigen-binding portion or variant can have an antigen-binding region that comprises at least a portion of at least one light chain CDR (i.e., CDR1, CDR2 and/or CDR3) having the amino acid sequence of the corresponding CDRs 1, 2 and/or 3.

Such antibodies can be prepared by chemically joining together the various portions (e.g., CDRs, framework) of the antibody using conventional techniques, by preparing and expressing a (i.e., one or more) nucleic acid molecule that encodes the antibody using conventional techniques of recombinant DNA technology or by using any other suitable method.

In one embodiment, an anti-IL12/23p40 antibody useful for the invention is a monoclonal antibody, preferably a human mAb, comprising heavy chain complementarity determining regions (CDRs) HCDR1, HCDR2, and HCDR3 of SEQ ID NOs: 1, 2, and 3, respectively; and light chain CDRs LCDR1, LCDR2, and LCDR3, of SEQ ID NOs: 4, 5, and 6, respectively.

The anti-IL12/23p40 antibody can comprise at least one of a heavy or light chain variable region having a defined amino acid sequence. For example, in a preferred embodiment, the anti-IL12/23p40 antibody comprises an anti-IL12/23p40 antibody with a heavy chain variable region comprising an amino acid sequence at least 85%, preferably at least 90%, more preferably at least 95%, and most preferably 100% identical to SEQ ID NO:7, and a light chain variable region comprising an amino acid sequence at least 85%, preferably at least 90%, more preferably at least 95%, and most preferably 100% identical to SEQ ID NO:8.

The anti-IL12/23p40 antibody can also comprise at least one of a heavy or light chain having a defined amino acid sequence. In another preferred embodiment, the anti-IL12/23p40 antibody comprises a heavy chain comprising an amino acid sequence at least 85%, preferably at least 90%, more preferably at least 95%, and most preferably 100% identical to SEQ ID NO:10, and a light chain variable region comprising an amino acid sequence at least 85%, preferably at least 90%, more preferably at least 95%, and most preferably 100% identical to SEQ ID NO:11.

Preferably, the anti-IL12/23p40 antibody is ustekinumab (Stelara®), comprising a heavy chain having the amino acid sequence of SEQ ID NO: 10 and a light chain comprising the amino acid sequence of SEQ ID NO: 11. Other examples of anti-IL12/23p40 antibodies useful for the invention include, but are not limited to, Briakinumab (ABT-874, Abbott) and other antibodies described in U.S. Pat. Nos. 6,914,128, 7,247,711, 7,700,739, the entire contents of which are incorporated herein by reference).

The invention also relates to antibodies, antigen-binding fragments, immunoglobulin chains and CDRs comprising amino acids in a sequence that is substantially the same as an amino acid sequence described herein. Preferably, such antibodies or antigen-binding fragments and antibodies comprising such chains or CDRs can bind human IL12/23p40 or IL-23 with high affinity (e.g., KD less than or equal to about 10⁻⁹M). Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include, without limitation, replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T.

Antibodies that bind to human IL-12/IL-23p40 or IL-23 and that comprise a defined heavy or light chain variable region can be prepared using suitable methods, such as phage display (Katsube, Y., et al., Int J Mol. Med, 1(5):863-868 (1998)) or methods that employ transgenic animals, as known in the art and/or as described herein. For example, a transgenic mouse, comprising a functionally rearranged human immunoglobulin heavy chain transgene and a transgene comprising DNA from a human immunoglobulin light chain locus that can undergo functional rearrangement, can be immunized with human IL12/23p40 or IL-23 or a fragment thereof to elicit the production of antibodies. If desired, the antibody producing cells can be isolated and hybridomas or other immortalized antibody-producing cells can be prepared as described herein and/or as known in the art. Alternatively, the antibody, specified portion or variant can be expressed using the encoding nucleic acid or portion thereof in a suitable host cell.

An anti-IL12/23p40 antibody used in the method of the present invention can include one or more amino acid substitutions, deletions or additions, either from natural mutations or human manipulation, as specified herein.

The number of amino acid substitutions a skilled artisan would make depends on many factors, including those described above. Generally speaking, the number of amino acid substitutions, insertions or deletions for any given anti-IL12/23p40 antibody, fragment or variant will not be more than 40, 30, 20, 19, 18, 17, 16, 15, 14, 13, 12, 11, 10, 9, 8, 7, 6, 5, 4, 3, 2, 1, such as 1-30 or any range or value therein, as specified herein.

Amino acids in an anti-IL12/23p40 antibody that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to, at least one IL12/23p40 or IL-23 neutralizing activity. Sites that are critical for antibody binding can also be identified by structural analysis, such as crystallization, nuclear magnetic resonance or photoaffinity labeling (Smith, et al., J. Mol. Biol. 224:899-904 (1992) and de Vos, et al., Science 255:306-312 (1992)).

Anti-IL12/23p40 antibodies can include, but are not limited to, at least one portion, sequence or combination selected from 5 to all of the contiguous amino acids of at least one of SEQ ID NOs 1, 2, 3, 4, 5, 6, 7, 8, 10, or 11.

Anti-IL12/23p40 antibodies or specified portions or variants can include, but are not limited to, at least one portion, sequence or combination selected from at least 3-5 contiguous amino acids of the SEQ ID NOs above; 5-17 contiguous amino acids of the SEQ ID NOs above, 5-10 contiguous amino acids of the SEQ ID NOs above, 5-11 contiguous amino acids of the SEQ ID NOs above, 5-7 contiguous amino acids of the SEQ ID NOs above; 5-9 contiguous amino acids of the SEQ ID NOs above.

An anti-IL12/23p40 antibody can further optionally comprise a polypeptide of at least one of 70-100% of 5, 17, 10, 11, 7, 9, 119, 108, 449, or 214 contiguous amino acids of the SEQ ID NOs above. In one embodiment, the amino acid sequence of an immunoglobulin chain, or portion thereof (e.g., variable region, CDR) has about 70-100% identity (e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or any range or value therein) to the amino acid sequence of the corresponding chain of at least one of the SEQ ID NOs above. For example, the amino acid sequence of a light chain variable region can be compared with the sequence of the SEQ ID NOs above, or the amino acid sequence of a heavy chain CDR3 can be compared with the SEQ ID NOs above. Preferably, 70-100% amino acid identity (i.e., 90, 91, 92, 93, 94, 95, 96, 97, 98, 99, 100 or any range or value therein) is determined using a suitable computer algorithm, as known in the art.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). In addition, values for percentage identity can be obtained from amino acid and nucleotide sequence alignments generated using the default settings for the AlignX component of Vector NTI Suite 8.0 (Informax, Frederick, Md.).

Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215:403-410 (1990)). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm can also be used to determine identity.

Exemplary heavy chain and light chain variable regions sequences and portions thereof are provided in the SEQ ID NOs above. The antibodies of the present invention, or specified variants thereof, can comprise any number of contiguous amino acid residues from an antibody of the present invention, wherein that number is selected from the group of integers consisting of from 10-100% of the number of contiguous residues in an anti-IL12/23p40 antibody. Optionally, this subsequence of contiguous amino acids is at least about 10, 20, 30, 40, 50, 60, 70, 80, 90, 100, 110, 120, 130, 140, 150, 160, 170, 180, 190, 200, 210, 220, 230, 240, 250 or more amino acids in length, or any range or value therein. Further, the number of such subsequences can be any integer selected from the group consisting of from 1 to 20, such as at least 2, 3, 4, or 5.

As those of skill will appreciate, the present invention includes at least one biologically active antibody of the present invention. Biologically active antibodies have a specific activity at least 20%, 30%, or 40%, and, preferably, at least 50%, 60%, or 70%, and, most preferably, at least 80%, 90%, or 95%-100% or more (including, without limitation, up to 10 times the specific activity) of that of the native (non-synthetic), endogenous or related and known antibody. Methods of assaying and quantifying measures of enzymatic activity and substrate specificity are well known to those of skill in the art.

In another aspect, the invention relates to human antibodies and antigen-binding fragments, as described herein, which are modified by the covalent attachment of an organic moiety. Such modification can produce an antibody or antigen-binding fragment with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified antibodies and antigen-binding fragments can comprise one or more organic moieties that are covalently bonded, directly or indirectly, to the antibody. Each organic moiety that is bonded to an antibody or antigen-binding fragment of the invention can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, an antibody modified by the covalent attachment of polylysine is encompassed by the invention. Hydrophilic polymers suitable for modifying antibodies of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the antibody of the invention has a molecular weight of about 800 to about 150,000 Daltons as a separate molecular entity. For example, PEG5000 and PEG20,000, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N, N-carbonyl diimidazole) on a fatty acid or fatty acid ester can be coupled to a hydroxyl group on a polymer.

Fatty acids and fatty acid esters suitable for modifying antibodies of the invention can be saturated or can contain one or more units of unsaturation. Fatty acids that are suitable for modifying antibodies of the invention include, for example, n-dodecanoate (C12, laurate), n-tetradecanoate (C14, myristate), n-octadecanoate (C18, stearate), n-eicosanoate (C20, arachidate), n-docosanoate (C22, behenate), n-triacontanoate (C30), n-tetracontanoate (C40), cis-Δ9-octadecanoate (C18, oleate), all cis-Δ5,8,11,14-eicosatetraenoate (C20, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably, one to about six, carbon atoms.

The modified human antibodies and antigen-binding fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups, such as tosylate, mesylate, halo (chloro, bromo, fluoro, iodo), N-hydroxysuccinimidyl esters (NETS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example, a divalent C1-C12 group wherein one or more carbon atoms can be replaced by a heteroatom, such as oxygen, nitrogen or sulfur. Suitable linker moieties include, for example, tetraethylene glycol, —(CH2)3-, —NH—(CH2)6-NH—, —(CH2)2-NH— and —CH2-O—CH2-CH2-O—CH2-CH2-O—CH—NH—.

Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate, as described, or can be reacted with maleic anhydride and the resulting product cyclized to produce an activated maleimido derivative of the fatty acid. (See, for example, Thompson, et al., WO 92/16221, the entire teachings of which are incorporated herein by reference.)

The modified antibodies can be produced by reacting a human antibody or antigen-binding fragment with a modifying agent. For example, the organic moieties can be bonded to the antibody in a non-site specific manner by employing an amine-reactive modifying agent, for example, an NHS ester of PEG. Modified human antibodies or antigen-binding fragments can also be prepared by reducing disulfide bonds (e.g., intra-chain disulfide bonds) of an antibody or antigen-binding fragment. The reduced antibody or antigen-binding fragment can then be reacted with a thiol-reactive modifying agent to produce the modified antibody of the invention. Modified human antibodies and antigen-binding fragments comprising an organic moiety that is bonded to specific sites of an antibody of the present invention can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6(10):2233-2241 (1997); Itoh et al., Bioorg. Chem., 24(1): 59-68 (1996); Capellas et al., Biotechnol. Bioeng., 56(4):456-463 (1997)), and the methods described in Hermanson, G. T., Bioconjugate Techniques, Academic Press: San Diego, Calif. (1996).

The method of the present invention also uses an anti-IL12/23p40 antibody composition comprising at least one, at least two, at least three, at least four, at least five, at least six or more anti-IL12/23p40 antibodies thereof, as described herein and/or as known in the art that are provided in a non-naturally occurring composition, mixture or form. Such compositions comprise non-naturally occurring compositions comprising at least one or two full length, C- and/or N-terminally deleted variants, domains, fragments, or specified variants, of the anti-IL12/23p40 antibody amino acid sequence selected from the group consisting of 70-100% of the contiguous amino acids of the SEQ ID NOs above, or specified fragments, domains or variants thereof. Preferred anti-IL12/23p40 antibody compositions include at least one or two full length, fragments, domains or variants as at least one CDR or LBP containing portions of the anti-IL12/23p40 antibody sequence described herein, for example, 70-100% of the SEQ ID NOs above, or specified fragments, domains or variants thereof. Further preferred compositions comprise, for example, 40-99% of at least one of 70-100% of the SEQ ID NOs above, etc., or specified fragments, domains or variants thereof. Such composition percentages are by weight, volume, concentration, molarity, or molality as liquid or dry solutions, mixtures, suspension, emulsions, particles, powder, or colloids, as known in the art or as described herein.

Antibody Compositions Comprising Further Therapeutically Active Ingredients

The antibody compositions used in the method of the invention can optionally further comprise an effective amount of at least one compound or protein selected from at least one of an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplastic, an immunomodulation drug, an ophthalmic, otic or nasal drug, a topical drug, a nutritional drug or the like. Such drugs are well known in the art, including formulations, indications, dosing and administration for each presented herein (see, e.g., Nursing 2001 Handbook of Drugs, 21st edition, Springhouse Corp., Springhouse, P A, 2001; Health Professional's Drug Guide 2001, ed., Shannon, Wilson, Stang, Prentice-Hall, Inc, Upper Saddle River, N.J.; Pharmcotherapy Handbook, Wells et al., ed., Appleton & Lange, Stamford, Conn., each entirely incorporated herein by reference).

By way of example of the drugs that can be combined with the antibodies for the method of the present invention, the anti-infective drug can be at least one selected from amebicides or at least one antiprotozoals, anthelmintics, antifungals, antimalarials, antituberculotics or at least one antileprotics, aminoglycosides, penicillins, cephalosporins, tetracyclines, sulfonamides, fluoroquinolones, antivirals, macrolide anti-infectives, and miscellaneous anti-infectives. The hormonal drug can be at least one selected from corticosteroids, androgens or at least one anabolic steroid, estrogen or at least one progestin, gonadotropin, antidiabetic drug or at least one glucagon, thyroid hormone, thyroid hormone antagonist, pituitary hormone, and parathyroid-like drug. The at least one cephalosporin can be at least one selected from cefaclor, cefadroxil, cefazolin sodium, cefdinir, cefepime hydrochloride, cefixime, cefmetazole sodium, cefonicid sodium, cefoperazone sodium, cefotaxime sodium, cefotetan disodium, cefoxitin sodium, cefpodoxime proxetil, cefprozil, ceftazidime, ceftibuten, ceftizoxime sodium, ceftriaxone sodium, cefuroxime axetil, cefuroxime sodium, cephalexin hydrochloride, cephalexin monohydrate, cephradine, and loracarbef.

The at least one coricosteroid can be at least one selected from betamethasone, betamethasone acetate or betamethasone sodium phosphate, betamethasone sodium phosphate, cortisone acetate, dexamethasone, dexamethasone acetate, dexamethasone sodium phosphate, fludrocortisone acetate, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, prednisolone, prednisolone acetate, prednisolone sodium phosphate, prednisolone tebutate, prednisone, triamcinolone, triamcinolone acetonide, and triamcinolone diacetate. The at least one androgen or anabolic steroid can be at least one selected from danazol, fluoxymesterone, methyltestosterone, nandrolone decanoate, nandrolone phenpropionate, testosterone, testosterone cypionate, testosterone enanthate, testosterone propionate, and testosterone transdermal system.

The at least one immunosuppressant can be at least one selected from azathioprine, basiliximab, cyclosporine, daclizumab, lymphocyte immune globulin, muromonab-CD3, mycophenolate mofetil, mycophenolate mofetil hydrochloride, sirolimus, 6-mercaptopurine, methotrexate, mizoribine, and tacrolimus.

The at least one local anti-infective can be at least one selected from acyclovir, amphotericin B, azelaic acid cream, bacitracin, butoconazole nitrate, clindamycin phosphate, clotrimazole, econazole nitrate, erythromycin, gentamicin sulfate, ketoconazole, mafenide acetate, metronidazole (topical), miconazole nitrate, mupirocin, naftifine hydrochloride, neomycin sulfate, nitrofurazone, nystatin, silver sulfadiazine, terbinafine hydrochloride, terconazole, tetracycline hydrochloride, tioconazole, and tolnaftate. The at least one scabicide or pediculicide can be at least one selected from crotamiton, lindane, permethrin, and pyrethrins. The at least one topical corticosteroid can be at least one selected from betamethasone dipropionate, betamethasone valerate, clobetasol propionate, desonide, desoximetasone, dexamethasone, dexamethasone sodium phosphate, diflorasone diacetate, fluocinolone acetonide, fluocinonide, flurandrenolide, fluticasone propionate, halcionide, hydrocortisone, hydrocortisone acetate, hydrocortisone butyrate, hydrocorisone valerate, mometasone furoate, and triamcinolone acetonide. (See, e.g., pp. 1098-1136 of Nursing 2001 Drug Handbook.)

Anti-IL12/23p40 antibody compositions can further comprise at least one of any suitable and effective amount of a composition or pharmaceutical composition comprising at least one anti-IL12/23p40 antibody contacted or administered to a cell, tissue, organ, animal or subject in need of such modulation, treatment or therapy, optionally further comprising at least one selected from at least one TNF antagonist (e.g., but not limited to a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-1 or TBP-II), nerelimonmab, infliximab, eternacept, CDP-571, CDP-870, afelimomab, lenercept, and the like), an antirheumatic (e.g., methotrexate, auranofin, aurothioglucose, azathioprine, etanercept, gold sodium thiomalate, hydroxychloroquine sulfate, leflunomide, sulfasalzine), an immunization, an immunoglobulin, an immunosuppressive (e.g., azathioprine, basiliximab, cyclosporine, daclizumab), a cytokine or a cytokine antagonist. Non-limiting examples of such cytokines include, but are not limited to, any of IL-1 to IL-23 et al. (e.g., IL-1, IL-2, etc.). Suitable dosages are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

Anti-IL12/23p40 antibody compounds, compositions or combinations used in the method of the present invention can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, Pa.) 1990. Pharmaceutically acceptable carriers can be routinely selected that are suitable for the mode of administration, solubility and/or stability of the anti-IL12/23p40, fragment or variant composition as well known in the art or as described herein.

Pharmaceutical excipients and additives useful in the present composition include, but are not limited to, proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Exemplary protein excipients include serum albumin, such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Carbohydrate excipients suitable for use in the invention include, for example, monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present invention are mannitol, trehalose, and raffinose.

Anti-IL12/23p40 antibody compositions can also include a buffer or a pH adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers for use in the present compositions are organic acid salts, such as citrate.

Additionally, anti-IL12/23p40 antibody compositions can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the anti-IL12/23p40 antibody, portion or variant compositions according to the invention are known in the art, e.g., as listed in “Remington: The Science & Practice of Pharmacy,” 19th ed., Williams & Williams, (1995), and in the “Physician's Desk Reference,” 52nd ed., Medical Economics, Montvale, N.J. (1998), the disclosures of which are entirely incorporated herein by reference. Preferred carrier or excipient materials are carbohydrates (e.g., saccharides and alditols) and buffers (e.g., citrate) or polymeric agents. An exemplary carrier molecule is the mucopolysaccharide, hyaluronic acid, which can be useful for intraarticular delivery.

Formulations

As noted above, the invention provides for stable formulations, which preferably comprise a phosphate buffer with saline or a chosen salt, as well as preserved solutions and formulations containing a preservative as well as multi-use preserved formulations suitable for pharmaceutical or veterinary use, comprising at least one anti-IL12/23p40 antibody in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1, 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

As noted above, the method of the invention uses an article of manufacture, comprising packaging material and at least one vial comprising a solution of at least one anti-IL12/23p40 antibody with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further uses an article of manufacture, comprising packaging material, a first vial comprising lyophilized anti-IL12/23p40 antibody, and a second vial comprising an aqueous diluent of prescribed buffer or preservative, wherein said packaging material comprises a label that instructs a subject to reconstitute the anti-IL12/23p40 antibody in the aqueous diluent to form a solution that can be held over a period of twenty-four hours or greater.

The anti-IL12/23p40 antibody used in accordance with the present invention can be produced by recombinant means, including from mammalian cell or transgenic preparations, or can be purified from other biological sources, as described herein or as known in the art.

The range of the anti-IL12/23p40 antibody includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

Preferably, the aqueous diluent optionally further comprises a pharmaceutically acceptable preservative. Preferred preservatives include those selected from the group consisting of phenol, m-cresol, p-cresol, o-cresol, chlorocresol, benzyl alcohol, alkylparaben (methyl, ethyl, propyl, butyl and the like), benzalkonium chloride, benzethonium chloride, sodium dehydroacetate and thimerosal, or mixtures thereof. The concentration of preservative used in the formulation is a concentration sufficient to yield an anti-microbial effect. Such concentrations are dependent on the preservative selected and are readily determined by the skilled artisan.

Other excipients, e.g., isotonicity agents, buffers, antioxidants, and preservative enhancers, can be optionally and preferably added to the diluent. An isotonicity agent, such as glycerin, is commonly used at known concentrations. A physiologically tolerated buffer is preferably added to provide improved pH control. The formulations can cover a wide range of pHs, such as from about pH 4 to about pH 10, and preferred ranges from about pH 5 to about pH 9, and a most preferred range of about 6.0 to about 8.0. Preferably, the formulations of the present invention have a pH between about 6.8 and about 7.8. Preferred buffers include phosphate buffers, most preferably, sodium phosphate, particularly, phosphate buffered saline (PBS).

Other additives, such as a pharmaceutically acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) or non-ionic surfactants, such as polysorbate 20 or 80 or poloxamer 184 or 188, Pluronic® polyls, other block co-polymers, and chelators, such as EDTA and EGTA, can optionally be added to the formulations or compositions to reduce aggregation. These additives are particularly useful if a pump or plastic container is used to administer the formulation. The presence of pharmaceutically acceptable surfactant mitigates the propensity for the protein to aggregate.

The formulations can be prepared by a process which comprises mixing at least one anti-IL12/23p40 antibody and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing the at least one anti-IL12/23p40 antibody and preservative in an aqueous diluent is carried out using conventional dissolution and mixing procedures. To prepare a suitable formulation, for example, a measured amount of at least one anti-IL12/23p40 antibody in buffered solution is combined with the desired preservative in a buffered solution in quantities sufficient to provide the protein and preservative at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The formulations can be provided to subjects as clear solutions or as dual vials comprising a vial of lyophilized anti-IL12/23p40 antibody that is reconstituted with a second vial containing water, a preservative and/or excipients, preferably, a phosphate buffer and/or saline and a chosen salt, in an aqueous diluent. Either a single solution vial or dual vial requiring reconstitution can be reused multiple times and can suffice for a single or multiple cycles of subject treatment and thus can provide a more convenient treatment regimen than currently available.

The present articles of manufacture are useful for administration over a period ranging from immediate to twenty-four hours or greater. Accordingly, the presently claimed articles of manufacture offer significant advantages to the subject. Formulations of the invention can optionally be safely stored at temperatures of from about 2° C. to about 40° C. and retain the biologically activity of the protein for extended periods of time, thus allowing a package label indicating that the solution can be held and/or used over a period of 6, 12, 18, 24, 36, 48, 72, or 96 hours or greater. If preserved diluent is used, such label can include use up to 1-12 months, one-half, one and a half, and/or two years.

The solutions of anti-IL12/23p40 antibody can be prepared by a process that comprises mixing at least one antibody in an aqueous diluent. Mixing is carried out using conventional dissolution and mixing procedures. To prepare a suitable diluent, for example, a measured amount of at least one antibody in water or buffer is combined in quantities sufficient to provide the protein and, optionally, a preservative or buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed products can be provided to subjects as clear solutions or as dual vials comprising a vial of lyophilized anti-IL12/23p40 antibody that is reconstituted with a second vial containing the aqueous diluent. Either a single solution vial or dual vial requiring reconstitution can be reused multiple times and can suffice for a single or multiple cycles of subject treatment and thus provides a more convenient treatment regimen than currently available.

The claimed products can be provided indirectly to subjects by providing to pharmacies, clinics, or other such institutions and facilities, clear solutions or dual vials comprising a vial of lyophilized anti-IL12/23p40 antibody that is reconstituted with a second vial containing the aqueous diluent. The clear solution in this case can be up to one liter or even larger in size, providing a large reservoir from which smaller portions of the at least one antibody solution can be retrieved one or multiple times for transfer into smaller vials and provided by the pharmacy or clinic to their customers and/or subjects.

Recognized devices comprising single vial systems include pen-injector devices for delivery of a solution, such as BD Pens, BD Autojector®, Humaject®, NovoPen®, B-D® Pen, OnePress® (SelfDose®), AutoPen®, and OptiPen®, GenotropinPen®, Genotronorm Pen®, Humatro Pen®, Reco-Pen®, Roferon Pen®, Biojector®, Iject®, J-tip Needle-Free Injector®, Intraject®, Medi-Ject®, Smartject® e.g., as made or developed by Becton Dickensen (Franklin Lakes, N.J., www.bectondickenson.com), Disetronic (Burgdorf, Switzerland, www.disetronic.com; Bioject, Portland, Oreg. (www.bioject.com); National Medical Products, Weston Medical (Peterborough, UK, www.weston-medical.com), Medi-Ject Corp (Minneapolis, Minn., www.mediject.com), and similarly suitable devices. Recognized devices comprising a dual vial system include those pen-injector systems for reconstituting a lyophilized drug in a cartridge for delivery of the reconstituted solution, such as the HumatroPen®. Examples of other devices suitable include pre-filled syringes, auto-injectors, needle free injectors, and needle free IV infusion sets.

The products can include packaging material. The packaging material provides, in addition to the information required by the regulatory agencies, the conditions under which the product can be used. The packaging material of the present invention provides instructions to the subject, as applicable, to reconstitute the at least one anti-IL12/23p40 antibody in the aqueous diluent to form a solution and to use the solution over a period of 2-24 hours or greater for the two vial, wet/dry, product. For the single vial, solution product, pre-filled syringe or auto-injector, the label indicates that such solution can be used over a period of 2-24 hours or greater. The products are useful for human pharmaceutical product use.

The formulations used in the method of the present invention can be prepared by a process that comprises mixing an anti-IL12/23p40 and a selected buffer, preferably, a phosphate buffer containing saline or a chosen salt. Mixing the anti-IL12/23p40 antibody and buffer in an aqueous diluent is carried out using conventional dissolution and mixing procedures. To prepare a suitable formulation, for example, a measured amount of at least one antibody in water or buffer is combined with the desired buffering agent in water in quantities sufficient to provide the protein and buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The method of the invention provides pharmaceutical compositions comprising various formulations useful and acceptable for administration to a human or animal subject. Such pharmaceutical compositions are prepared using water at “standard state” as the diluent and routine methods well known to those of ordinary skill in the art. For example, buffering components such as histidine and histidine monohydrochloride hydrate, can be provided first followed by the addition of an appropriate, non-final volume of water diluent, sucrose and polysorbate 80 at “standard state.” Isolated antibody can then be added. Last, the volume of the pharmaceutical composition is adjusted to the desired final volume under “standard state” conditions using water as the diluent. Those skilled in the art will recognize a number of other methods suitable for the preparation of the pharmaceutical compositions.

The pharmaceutical compositions can be aqueous solutions or suspensions comprising the indicated mass of each constituent per unit of water volume or having an indicated pH at “standard state.” As used herein, the term “standard state” means a temperature of 25° C.+/−2° C. and a pressure of 1 atmosphere. The term “standard state” is not used in the art to refer to a single art recognized set of temperatures or pressure, but is instead a reference state that specifies temperatures and pressure to be used to describe a solution or suspension with a particular composition under the reference “standard state” conditions. This is because the volume of a solution is, in part, a function of temperature and pressure. Those skilled in the art will recognize that pharmaceutical compositions equivalent to those disclosed here can be produced at other temperatures and pressures. Whether such pharmaceutical compositions are equivalent to those disclosed here should be determined under the “standard state” conditions defined above (e.g. 25° C.+/−2° C. and a pressure of 1 atmosphere).

Importantly, such pharmaceutical compositions can contain component masses “about” a certain value (e.g. “about 0.53 mg L-histidine”) per unit volume of the pharmaceutical composition or have pH values about a certain value. A component mass present in a pharmaceutical composition or pH value is “about” a given numerical value if the isolated antibody present in the pharmaceutical composition is able to bind a peptide chain while the isolated antibody is present in the pharmaceutical composition or after the isolated antibody has been removed from the pharmaceutical composition (e.g., by dilution). Stated differently, a value, such as a component mass value or pH value, is “about” a given numerical value when the binding activity of the isolated antibody is maintained and detectable after placing the isolated antibody in the pharmaceutical composition.

Competition binding analysis is performed to determine if the IL12/23p40 mAbs bind to similar or different epitopes and/or compete with each other. Abs are individually coated on ELISA plates. Competing mAbs are added, followed by the addition of biotinylated hrIL-12 or IL-23. For positive control, the same mAb for coating can be used as the competing mAb (“self-competition”). IL12/IL23p40 or IL-23 binding is detected using streptavidin. These results demonstrate whether the mAbs recognize similar or partially overlapping epitopes on IL12/23p40 or IL-23.

In one embodiment of the pharmaceutical compositions, the isolated antibody concentration is from about 77 to about 104 mg per ml of the pharmaceutical composition. In another embodiment of the pharmaceutical compositions the pH is from about 5.5 to about 6.5.

The stable or preserved formulations can be provided to subjects as clear solutions or as dual vials comprising a vial of lyophilized at least one anti-IL12/23p40 that is reconstituted with a second vial containing a preservative or buffer and excipients in an aqueous diluent. Either a single solution vial or dual vial requiring reconstitution can be reused multiple times and can suffice for a single or multiple cycles of subject treatment and thus provides a more convenient treatment regimen than currently available.

Other formulations or methods of stabilizing the anti-IL12/23p40 can result in other than a clear solution of lyophilized powder comprising the antibody. Among non-clear solutions are formulations comprising particulate suspensions, said particulates being a composition containing the anti-IL12/23p40 in a structure of variable dimension and known variously as a microsphere, microparticle, nanoparticle, nanosphere, or liposome. Such relatively homogenous, essentially spherical, particulate formulations containing an active agent can be formed by contacting an aqueous phase containing the active agent and a polymer and a nonaqueous phase followed by evaporation of the nonaqueous phase to cause the coalescence of particles from the aqueous phase as taught in U.S. Pat. No. 4,589,330. Porous microparticles can be prepared using a first phase containing active agent and a polymer dispersed in a continuous solvent and removing said solvent from the suspension by freeze-drying or dilution-extraction-precipitation as taught in U.S. Pat. No. 4,818,542. Preferred polymers for such preparations are natural or synthetic copolymers or polymers selected from the group consisting of glelatin agar, starch, arabinogalactan, albumin, collagen, polyglycolic acid, polylactic aced, glycolide-L(−) lactide poly(episilon-caprolactone, poly(epsilon-caprolactone-CO-lactic acid), poly(epsilon-caprolactone-CO-glycolic acid), poly(B-hydroxy butyric acid), polyethylene oxide, polyethylene, poly(alkyl-2-cyanoacrylate), poly(hydroxyethyl methacrylate), polyamides, poly(amino acids), poly(2-hydroxyethyl DL-aspartamide), poly(ester urea), poly(L-phenylalanine/ethylene glycol/1,6-diisocyanatohexane) and poly(methyl methacrylate). Particularly preferred polymers are polyesters, such as polyglycolic acid, polylactic aced, glycolide-L(−) lactide poly(episilon-caprolactone, poly(epsilon-caprolactone-CO-lactic acid), and poly(epsilon-caprolactone-CO-glycolic acid. Solvents useful for dissolving the polymer and/or the active include: water, hexafluoroisopropanol, methylenechloride, tetrahydrofuran, hexane, benzene, or hexafluoroacetone sesquihydrate. The process of dispersing the active containing phase with a second phase can include pressure forcing said first phase through an orifice in a nozzle to affect droplet formation.

Dry powder formulations can result from processes other than lyophilization, such as by spray drying or solvent extraction by evaporation or by precipitation of a crystalline composition followed by one or more steps to remove aqueous or non-aqueous solvent. Preparation of a spray-dried antibody preparation is taught in U.S. Pat. No. 6,019,968. The antibody-based dry powder compositions can be produced by spray drying solutions or slurries of the antibody and, optionally, excipients, in a solvent under conditions to provide a respirable dry powder. Solvents can include polar compounds, such as water and ethanol, which can be readily dried. Antibody stability can be enhanced by performing the spray drying procedures in the absence of oxygen, such as under a nitrogen blanket or by using nitrogen as the drying gas. Another relatively dry formulation is a dispersion of a plurality of perforated microstructures dispersed in a suspension medium that typically comprises a hydrofluoroalkane propellant as taught in WO 9916419. The stabilized dispersions can be administered to the lung of a subject using a metered dose inhaler. Equipment useful in the commercial manufacture of spray dried medicaments are manufactured by Buchi Ltd. or Niro Corp.

An anti-IL12/23p40 antibody in either the stable or preserved formulations or solutions described herein, can be administered to a subject in accordance with the present invention via a variety of delivery methods including SC or IM injection; transdermal, pulmonary, transmucosal, implant, osmotic pump, cartridge, micro pump, or other means appreciated by the skilled artisan, as well-known in the art.

Therapeutic Applications

The present invention also provides a method for modulating or treating ulcerative colitis, in a cell, tissue, organ, animal, or subject, as known in the art or as described herein, using an anti-IL12/23p40 antibody of the present invention, e.g., administering or contacting the cell, tissue, organ, animal, or subject with a therapeutic effective amount of anti-IL12/23p40 antibody.

Any method of the present invention can comprise administering an effective amount of a composition or pharmaceutical composition comprising an anti-IL12/23p40 antibody to a cell, tissue, organ, animal or subject in need of such modulation, treatment or therapy. Such a method can optionally further comprise co-administration or combination therapy for treating such diseases or disorders, wherein the administering of anti-IL12/23p40 antibody, specified portion or variant thereof, further comprises administering, before concurrently, and/or after, at least one selected from at least one TNF antagonist (e.g., but not limited to, a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-1 or TBP-II), nerelimonmab, infliximab, eternacept (Enbrel™), adalimulab (Humira™), CDP-571, CDP-870, afelimomab, lenercept, and the like), an antirheumatic (e.g., methotrexate, auranofin, aurothioglucose, azathioprine, gold sodium thiomalate, hydroxychloroquine sulfate, leflunomide, sulfasalzine), a muscle relaxant, a narcotic, a non-steroid anti-inflammatory drug (NSAID) (e.g., 5-aminosalicylate), an analgesic, an anesthetic, a sedative, a local anesthetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropoietin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist. Suitable dosages are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, C A (2000); Nursing 2001 Handbook of Drugs, 21st edition, Springhouse Corp., Springhouse, P A, 2001; Health Professional's Drug Guide 2001, ed., Shannon, Wilson, Stang, Prentice-Hall, Inc, Upper Saddle River, N.J., each of which references are entirely incorporated herein by reference.

Therapeutic Treatments

Treatment of ulcerative colitis is affected by administering an effective amount or dosage of an anti-IL12/23p40 antibody composition in a subject in need thereof. The dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

In one exemplary regimen of providing safe and effective treatment of Crohn's disease in a subject in need thereof, a total dosage of about 130 mg of an anti-IL12/23p40 antibody is administered intravenously to the subject per administration. For example, the total volume of the composition administered is appropriately adjusted to provide to the subject the target dosage of the antibody at 80 mg, 90 mg, 100 mg, 110 mg, 120 mg, 130 mg, 140 mg, 150 mg, 160 mg, 170 mg or 180 mg per administration.

In another exemplary regimen of providing safe and effective treatment of severely active UC in a subject in need thereof, a total dosage of about 6.0 mg/kg±1.5 mg/kg of an anti-IL12/23p40 antibody is administered intravenously to the subject per administration. For example, the total volume of the composition administered is appropriately adjusted to provide to the subject the target dosage of the antibody at 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, or 9.0 mg/kg body weight of the subject per administration.

The total dosage of an anti-IL12/23p40 antibody to be administered to the subject per administration can be administered by intravenous infusion over a period of about 30 minutes to 180 minutes, preferably 60 minutes to 120 minutes, such as 30 minutes, 60 minutes, 90 minutes, 120 minutes, 150 minutes, or 180 minutes.

In yet another exemplary regimen of providing safe and effective treatment of severely active UC in a subject in need thereof, a total dosage of about 90 mg of an anti-IL12/23p40 antibody is administered subcutaneously to the subject per administration. For example, the total volume of the composition administered is appropriately adjusted to provide to the subject the target dosage of the antibody at 40 mg, 50 mg, 60 mg, 70 mg, 80 mg, 90 mg, 100 mg, 110 mg, 120 mg, 130 mg or 140 mg per administration. The target dosage per administration can be administered in a single subcutaneous injection or in multiple subcutaneous injections, such as 1, 2, 3, 4, 5, or more subcutaneous injections.

The total dosage of the anti-IL12/23p40 antibody can be administered once per day, once per week, once per month, once every six months, etc. for a period of one day, one week, one month, six months, 1 year, 2 years or longer. Multiple administrations of the anti-IL12/23p40 antibody, each at a total dosage of described herein, can be administered to a subject in need thereof.

Dosage forms (composition) suitable for internal administration generally contain from about 0.001 milligram to about 500 milligrams of active ingredient per unit or container.

For parenteral administration, the antibody can be formulated as a solution, suspension, emulsion, particle, powder, or lyophilized powder in association, or separately provided, with a pharmaceutically acceptable parenteral vehicle. Examples of such vehicles are water, saline, Ringer's solution, dextrose solution, and 1-10% human serum albumin. Liposomes and nonaqueous vehicles, such as fixed oils, can also be used. The vehicle or lyophilized powder can contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

Many known and developed modes can be used according to the present invention for administering pharmaceutically effective amounts of an IL12/23p40 antibody. Anti-IL12/23p40 antibodies of the present invention can be delivered in a carrier, as a solution, emulsion, colloid, or suspension, or as a dry powder, using any of a variety of devices and methods suitable for administration by inhalation or other modes described here within or known in the art.

Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthtetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446 entirely incorporated herein by reference.

Alternative Delivery

The invention further relates to the administration of an anti-IL12/23p40 antibody by parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal means. An anti-IL12/23p40 antibody composition can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement;” Hsieh, D. S., Eds., pp. 59-90 (Marcel Dekker, Inc. New York 1994, entirely incorporated herein by reference), or with oxidizing agents that enable the application of formulations containing proteins and peptides onto the skin (WO 98/53847), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

### EMBODIMENTS

The invention provides also the following non-limiting embodiments.
1. A method of treating Crohn's disease in a subject in need thereof, comprising:
   performing endoscopy on the subject prior to treatment to measure baseline simple endoscopic score for Crohn's disease (SES-CD) and baseline CDAI;
   administering to the subject a pharmaceutical composition comprising a clinically proven safe and clinically proven effective amount of an anti-IL-12/IL-23p40 antibody, wherein the antibody comprises a heavy chain variable region and a light chain variable region, the heavy chain variable region comprising: a complementarity determining region heavy chain 1 (CDRH1) amino acid sequence of SEQ ID NO:1; a CDRH2 amino acid sequence of SEQ ID NO:2; and a CDRH3 amino acid sequence of SEQ ID NO:3; and the light chain variable region comprising: a complementarity determining region light chain 1 (CDRL1) amino acid sequence of SEQ ID NO:4; a CDRL2 amino acid sequence of SEQ ID NO:5; and a CDRL3 amino acid sequence of SEQ ID NO:6 in an initial weight based IV dose of 6 mg of antibody per kg weight of the subject and a subcutaneous dose of 90 mg of antibody 8 weeks after administration of the initial dose;
   measuring (i) Crohn's-associated biomarkers selected from C-reactive protein (CRP) and/or faecal calprotectin (FCal) levels, and (ii) clinical symptoms selected from a CDAI and SES-CD of the subject 16 weeks after administration of the initial dose; and
   administering (iii) 90 mg of antibody by subcutaneous dose 16 weeks after administration of the initial dose and every four weeks after the subcutaneous dose at 16 weeks to subjects measured to have CDAI <220, less than 70 point improvement from baseline CDAI, CRP ≤10 mg/L and/or FCal ≤250 ug/g, or (iv) 90 mg of antibody by subcutaneous dose 16 weeks after administration of the initial dose and every eight weeks after the subcutaneous dose at 16 weeks to subjects measured to have less than a 25% improvement in SES-CD score versus baseline SES-CD score.
2. The method of embodiment 1, wherein the antibody comprises the heavy chain variable region of the amino acid sequence of SEQ ID NO:7 and the light chain variable region of the amino acid sequence of SEQ ID NO:8.
3. The method of embodiment 1, wherein the antibody comprises a heavy chain of the amino acid sequence of SEQ ID NO:10 and a light chain of the amino acid sequence of SEQ ID NO:11.
4. The method of any one of embodiments 1 to 3, wherein the antibody is administered intravenously to the subject, preferably at week 0 of the treatment, at a dosage of about 6.0 mg/kg body weight of the subject or 130 mg per administration.
5. The method of any one of embodiments 1 to 4, wherein the antibody is further administered subcutaneously to the subject, preferably at weeks 8 and 16 of the treatment and preferable every 4 or 8 weeks thereafter as determined by measured clinical parameters, at a dosage of about 90 mg per administration.
6. The method of any one of embodiments 1 to 5, wherein the subject had previously failed or were intolerant of at least one therapy selected from the group consisting of an anti-TNF, vedolizumab, corticosteroids, azathioprine (AZA), and 6 mercaptopurine (6 MP), or the subject had demonstrated corticosteroid dependence.
7. The method of any one of embodiments 1-6, wherein the pharmaceutical composition for intravenous administration further comprises a solution comprising 10 mM L-histidine, 8.5% (w/v) sucrose, 0.04% (w/v) polysorbate 80, 0.4 mg/mL L-methionine, and 20 μg/mL EDTA disodium salt, dehydrate, at pH 6.0.
8. The method of any one of embodiments 1-6, wherein the pharmaceutical composition for subcutaneous administration further comprises a solution comprising 6.7 mM L-histidine, 7.6% (w/v) sucrose, 0.004% (w/v) polysorbate 80, at pH 6.0.
   Having generally described the invention, the same will be more readily understood by reference to the following Example, which are provided by way of illustration and are not intended as limiting. Further details of the invention are illustrated by the following non-limiting Example. The disclosures of all citations in the specification are expressly incorporated herein by reference.

### EXAMPLE

### Example: Phase 3b Study of Ustekinumab for Treatment of Crohn's Disease Patients to Compare Treat to Target Strategy (T2T) to Standard of Care (SoC)

### Methods Summary

Adult patients with moderate to severely active Crohn's disease (CD) (CD activity index [CDAI] 220-450) and Simple Endoscopic Score in CD [SES-CD]≥3) who failed conventional therapy and/or one biologic were included. Patients received IV, weight-based ustekinumab (UST) of ˜6 mg/kg at Week 0 (baseline [BL]); subcutaneous (SC) UST 90 mg at Week 8. At Week 16, after an endoscopy performed on patients, CDAI 70 responders were randomized (1:1) to the T2T or SoC treatment arms. Patients in the T2T arm were assigned to SC UST q12w or q8w based on 25% improvement in SES-CD score vs BL. From Weeks 16-48, UST dose was further adjusted up to q4w if the following targets were not met: CDAI <220 and ≥70-point improvement from BL, and C-reactive protein (CRP)≤10 mg/L or faecal calprotectin (FCal) ≤250 μg/g. Patients who failed treatment target despite UST q4w were discontinued. In the SoC arm, UST dose was assigned by the investigator based on EU SmPC (q12w or q8w). Primary endpoint: endoscopic response at Week 48 (≥50% reduction in SES-CD score vs BL on centrally read endoscopies). Non-responder imputation (NRI) and last observation carried forward (LOCF) were used for missing dichotomous and continuous variables respectively. LOCF analysis was also a pre-planned sensitivity analysis for primary endpoint. All p-values reported are nominal.

### Results Summary

In total, 500 patients were enrolled. At Week 16, 441 achieved a CDAI 70 response and were randomized to T2T (n=220) or SoC (n=221); 75% and 86% in the T2T and SoC respectively, completed 48 Weeks. A numerically higher proportion of patients in the T2T arm vs SoC achieved the primary endpoint at Week 48: 37.7% vs 29.9% (p=0.0933). In a prespecified sensitivity analysis (LOCF) a significant difference was reached between arms: 40.0% (T2T) vs 30.8% (SoC) (p=0.0494 (LOCF]); at Week 48, high rates of clinical response were achieved in both T2T and SoC arm: 68.2% vs 77.8% (p=0.0212) (NRI)/89.5% vs 89.6% (LOCF; NS); clinical remission 61.4% vs 69.7% (NRI)/76.8% vs 78.3% (LOCF; NS); improvement of ≥50% in FCal 39.4% vs 46.5% (NRI)/63.1% vs 60.6% (LOCF; NS) and CRP levels 41.7% vs 53.3% p=0.032 (NRI)/53.2% vs 57.2% (LOCF, NS). See Tables for other endpoints. In the T2T and SoC arm, 59.2% (122/206) and 53.2% (116/218) of patients started on UST q12w; 59.8% (73/122) and 63.8% (74/116) of those were still on q12w at Week 48. Of patients who started on q8w, 40.5 (34/84) and 78.4% (80/102) remained on this regimen at Week 48 in T2T and SoC arms respectively. At Week 48 17% (35/206) of patients were on q4w dosing in the T2T arm. No new safety signals were reported.

A long-term extension (LTE; from Week 48 to Week 104) period was designed to explore the efficacy of a clinical symptoms, endoscopy, and the biomarker-driven dose adjustment (including de-escalation) algorithm. From Week 48, patients continued to receive subcutaneous ustekinumab in the LTE period, up to Week 104. The frequency of ustekinumab dosing with escalation/de-escalation between q12w/q8w/q4w was based on the following targets: endoscopic remission (Simple Endoscopic Score for CD [SES-CD] score ≤2) and corticosteroid (CS)-free clinical remission ([CDAI score of <150 points] of ≥16 weeks' duration) at Week 48; and later, on CS-free clinical remission and biomarker remission (C-reactive protein ≤10 mg/L and fecal calprotectin ≤250 μg/g) at 2 consecutive visits 8 weeks apart. Patients on q4w dosing failing to reach targets were discontinued. Presented results are non-responder imputation (NRI) analyses and are shown only for patients entering LTE (modified RAS [mRAS]).

Of 440 patients randomized to either T2T or SoC at Week 16, 74 dropped out before Week 48. Of the remaining 366 patients who completed Week 48, 43 discontinued and did not enter LTE; of these, 15 patients on q4w dosing discontinued as targets were unmet. At Week 48, 323 patients entered LTE (mRAS): 7.7% of patients were on q4w, 48.6% on q8w and 43.6% on q12w dosing. These proportions were 14.3%, 39.4%, and 46.3%, respectively, at Week 104/early dropout after excluding 8 untreated patients. A total of 20.1% of patients discontinued before completing Week 104. Overall, for 38.4% of patients, dose was escalated/de-escalated at least once during LTE with a similar proportion of patients receiving a dose escalation (22.9%) or de-escalation (19.2%). Clinical response and remission were observed in a high proportion of patients entering LTE at Week 48 (92.6% and 83.9% respectively) and remained high at Week 104 (70.9% and 68.4%, respectively). Over the course of LTE, the proportions of patients in endoscopic response and remission were 43.7% and 17.0% at entry to LTE vs 39.3% and 14.6%, respectively, at Week 104 (Table 4). No new safety signals were observed during LTE.

### Conclusion Summary

STARDUST is the first randomized T2T trial to use endoscopy at Week 16 to guide dose escalation in patients with CD. After 48 weeks of maintenance therapy with UST, a higher proportion of patients reached an endoscopic response in the T2T arm as compared to the SoC arm. T2T could be an additional tool for physicians to guide dosing regimen decisions with UST. Overall, high clinical remission and biomarker responses with UST were achieved in both arms with UST at Week 48. A similar proportion of patients (˜20%) underwent dose escalation/de-escalation during LTE as per the set targets; yet a majority of patients completed Week 104 on the label ustekinumab dosing. A majority of patients entering LTE and at Week 104 were in clinical response and remission. Flexible ustekinumab dosing enabled preservation of the proportions of patients in clinical and endoscopic response and remission during LTE. Ustekinumab had a favourable risk-benefit ratio at Week 104.

### Objective

To test the hypothesis that a maintenance strategy with UST based on:
Early endoscopy, followed by;
Regular assessment of biomarkers (fCal, CRP) and clinical symptoms (CDAI) with;
Subsequent adjustment of treatment to achieve target

is more successful in obtaining endoscopic improvement than a pragmatic maintenance strategy with analysis after 48 weeks of therapy with UST.

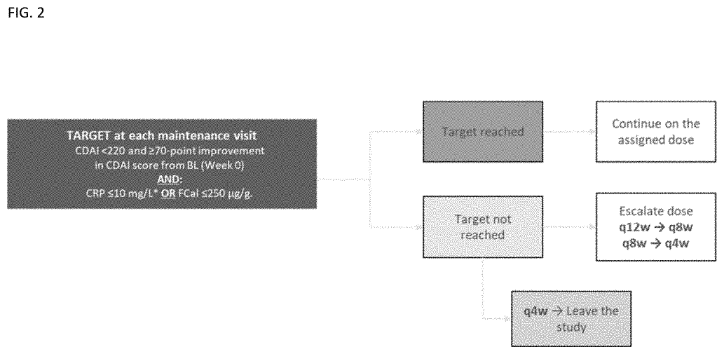

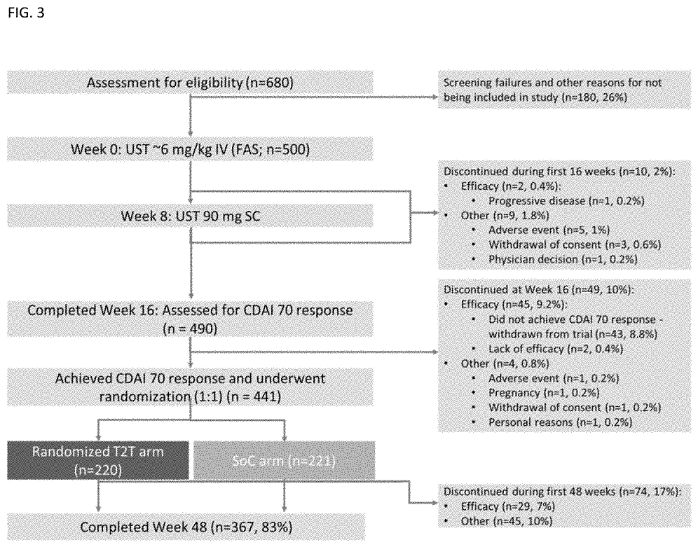

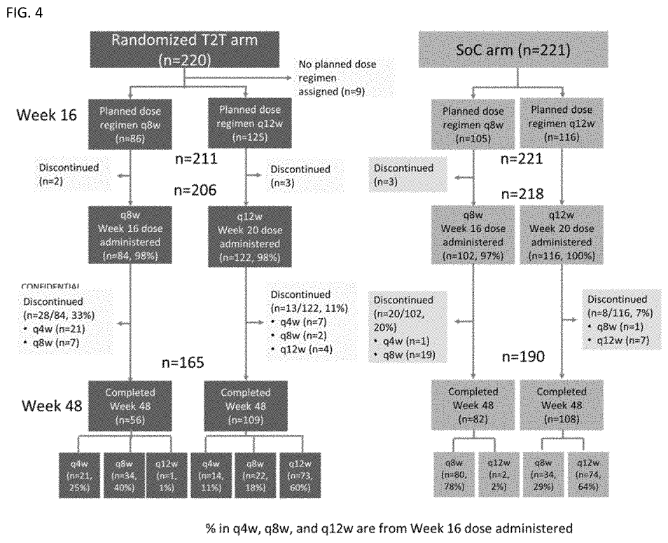

FIG. 2 shows the study design and dose adjustment criteria based on CDAI, CRP and FCal measurements. For patients who do not have elevated CRP at BL (i.e., CRP ≤2.87 mg/L at Week 0) in the presence of active disease, CRP is not considered a biomarker target for dose adjustment, therefore the treatment target for these patients will be the achievement of: CDAI <220 and ≥70-point improvement in CDAI score from BL (Week 0) AND FCal ≤250 μg/g.

Included in the trial were:
Patients (age ≥18 years) with moderate to severely active Crohn's disease (CDAI 220-450 and SES-CD ≥3) who had failed conventional therapy and/or one biologic agent were eligible to be enrolled in the trial
Eligible patients received a single dose of IV, weight-tiered, UST ˜6 mg/kg, followed by SC UST 90 mg at Week 8
At Week 16, CDAI 70 responders were randomized to the T2T or SoC treatment arm (1:1 ratio)
Key endpoints (NRI and LOCF imputation) analyzed at Weeks 8, 16, and 48
   Primary endpoint
      Endoscopic response (decrease from baseline in SES-CD score of ≥50%)
   Key secondary endpoints
      Overall Endoscopic remission (SES-CD score ≤2)
      Mucosal healing (complete absence of mucosal ulcerations in any ileocolonic segment)
      CDAI 70 response (an improvement of CDAI total score ≥70 points versus baseline)
      Clinical response (≥100-point reduction from the baseline CDAI total score, or a CDAI total score <150)
      Clinical remission (CDAI total score of <150 points
      Change from baseline in biomarkers (fCal and CRP)

Statistical analysis was performed as follows:
FAS included all enrolled patients who received at least one dose of UST
RAS included all patients who were randomized at Week 16 (CDAI 70 responders)
Non responder imputation (NRI) and last observation carried forward (LOCF) were used for missing dichotomous and continuous variables respectively. LOCF analysis was also a pre-planned sensitivity analysis for primary endpoint.
For dichotomous endpoints the CMH chi-square test is used to test between treatment groups. p-values (nominal) are based on the CMH test, 2-sided α level of 0.05, stratified by baseline SES-CD score (≤16, >16) and prior exposure to biologics (none or 1)
Continuous endpoints will be compared using an analysis of variance (ANOVA) or covariance (ANCOVA) with baseline value and stratification factors as a covariate. If the normality assumption is in question, an ANOVA or ANCOVA on the van der Waerden normal scores will be used
Time to event endpoints will be compared between treatment groups using the stratified log-rank test with prior exposure to biologic and baseline SES-CD score (≤16 or >16) as the stratification factors, unless otherwise specified

For the dose distribution in the T2T arm, 59% were on q12w at Week 16 and 41% were on q8w at Week 16 (n=206). At Week 48 in T2T, 36% were on q12w, 27% were on q8w and 17% were on q4w (with 20% discontinued). Of patients who started on q12w 59.8% (73/122) were still on q12w at Week 48. Of patients who started on q8w, 40.5 (34/84) remained on q8w at Week 48.

For the dose distribution in the SoC arm, 53% were on q12w at Week 16 and 47% were on q8w at Week 16 (n=218). At Week 48 in SoC, 35% were on q12w and 52% were on q8w (with 13% discontinued). Of patients who started on q12w, 63.8% (74/116) of were still on q12w at Week 48. Of patients who started on q8w, 78.4% (80/102) remained on this regimen at Week 48.

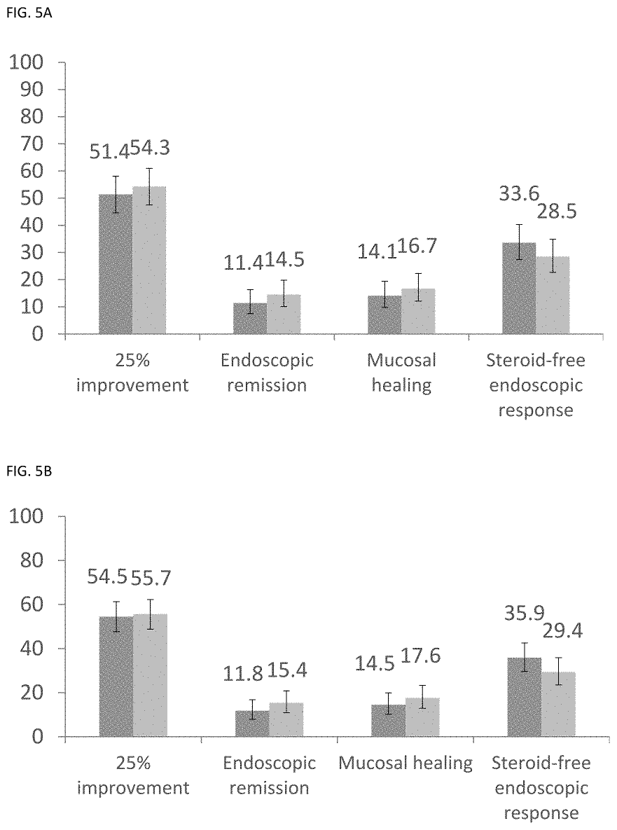

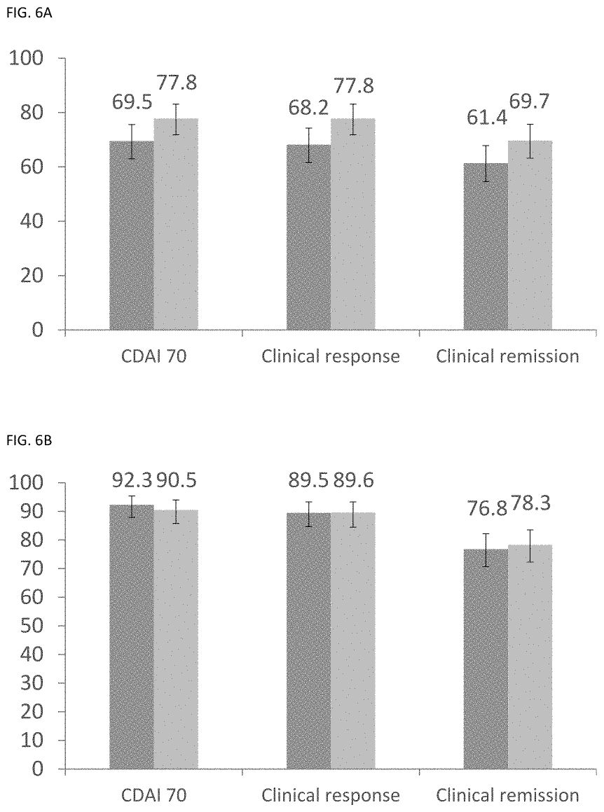

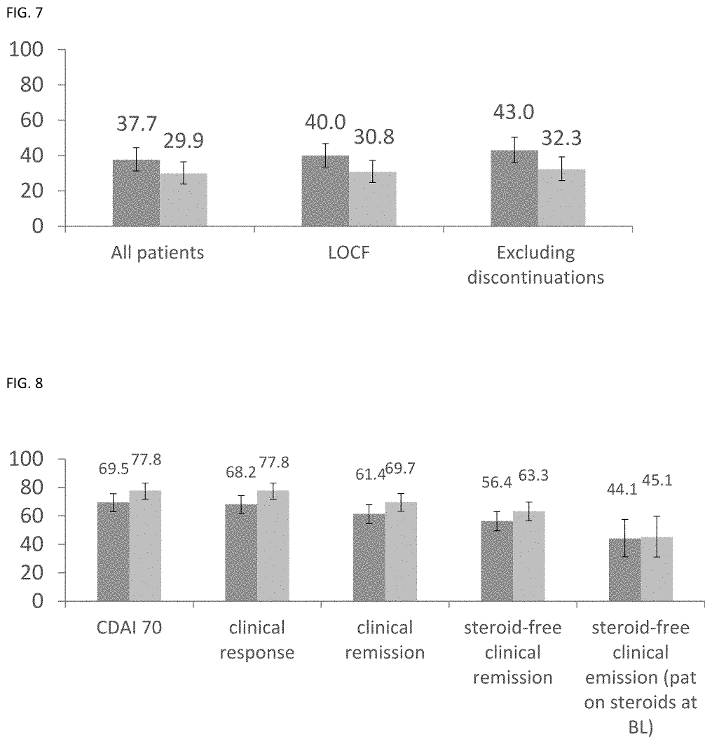

FIGS. 5A, 5B, 6A, 6B and 7 show endoscopic response measured with different variables. The Primary endpoint is Endoscopic response (SES-CD improvement ≥50%) at 48 weeks. This was numerically higher in the T2T vs SoC groups (NRI imputation) 37.7% vs. 29.9% p=0.09. LOCF or NRI that only included patients who discontinued the study for inefficacy: Significance in favour of T2T vs SoC group 40.0% vs 30.8% p<0.05 for LOCF, 43.0% vs 32.3% p=0.036 for NRI (incl. D/C of inefficacy only). The Secondary endpoints (LOCF) are Mean SES-CD change at w48 vs BL, % pts with ≥25% improvement in SES-CD, endoscopic remission and mucosal healing similar in T2T and SoC groups. Meaningful change of SES-CD vs BL reached at Week 16 for T2T, progressing up to Week 48.

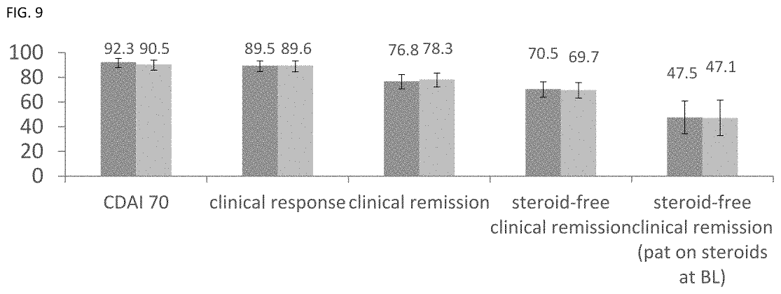

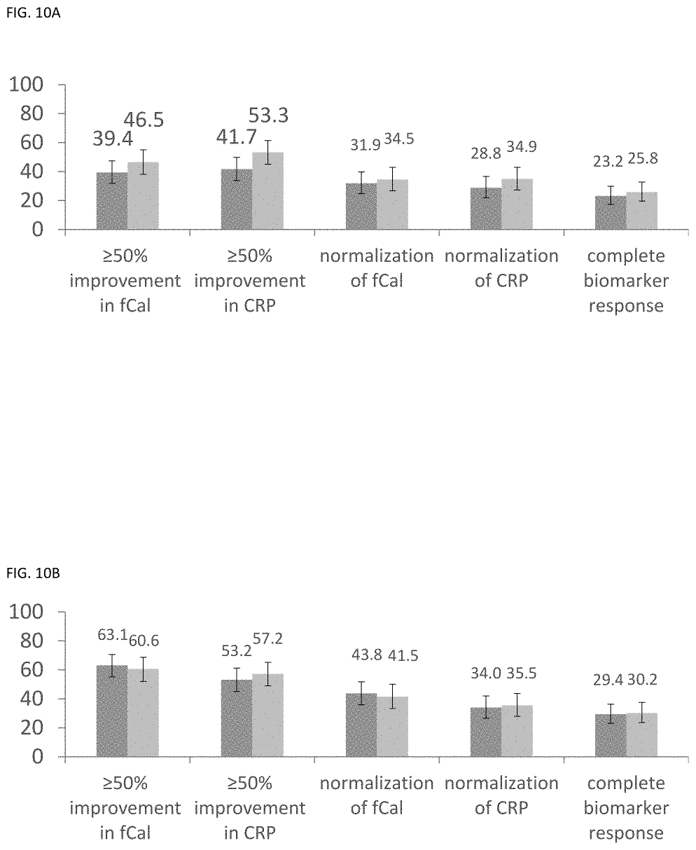

FIGS. 8 and 9 show clinical endpoints. Secondary endpoints at Week 48 are mean CDAI change vs baseline was similar in T2T and SoC; meaningful change vs BL reached at Week 8, progressing up to Week 48. Clinical response (CHANGE ≥100 or CDAI score <150), Clinical remission (CDAI score <150) and CDAI 70 response similar in both study arms.

FIGS. 10A and 10B show biomarker outcomes at Week 48 for RAS NRI and RAS LOCF. The Secondary endpoints are: Mean change vs baseline in fCal and CRP was similar in T2T and SoC at all timepoints. Meaningful change vs BL reached at Week 8, progressing up to Week 48. fCal and CRP achievement ≥50% improvement and normalisation in fCal and CRP levels and complete biomarkers response at 48 weeks were similar in both study arms.

TABLE 1
Patient disposition at Week 48 (RAS)

| | T2T arm | SoC arm | |
|---|---|---|---|
| | (n = 220) | (n = 221) | RAS (n = 441) |
| Completed Week 48* | 174 (79.1%) | 193 (87.3%) | 367 (83.2%) |
| Discontinued prematurely | 46 (20.9%) | 28 (12.7%) | 74 (16.8%) |
| in first 48 weeks of study | | | |
| Reasons for discontinuation | | | |
| Efficacy | 23 (10.5%) | 11 (5.2%) | 34 (7.7%) |
| Lack of efficacy | 21 (9.5%) | 8 (3.6%) | 29 (6.6%) |
| Disease relapse | 2 (0.9%) | 1 (0.5%) | 3 (0.7%) |
| Progressive disease | 0 | 1 (0.5%) | 1 (0.2%) |
| Received a disallowed | 0 | 1 (0.5%) | 1 (0.2%) |
| concomitant treatment | | | |
| Other | 23 (10.5%) | 17 (7.8%) | 40 (9.1%) |
| Withdrawal by subject | 12 (5.5%) | 4 (1.8%) | 16 (3.6%) |
| Adverse event | 6 (2.7%) | 10 (4.5%) | 16 (3.6%) |
| Death | 2 (0.9%) | 0 | 2 (0.5%) |
| Lost to follow-up | 1 (0.5%) | 0 | 1 (0.2%) |
| Physician decision | 1 (0.5%) | 2 (0.9%) | 3 (0.7%) |
| Pregnancy | 1 (0.5%) | 1 (0.5%) | 2 (0.5%) |

Completers include subjects with Early Termination Visit falling with in Week 48 Visit window. 75% and 86% in the T2T and SoC respectively, completed treatment for 48W

TABLE 2
Summary of demographics and baseline characteristics

| | T2T arm | SoC arm | RAS |
|---|---|---|---|
| | (n = 220) | (n = 221) | (n = 441) |
| Age, months, mean (SD) | 38.2 | 36.3 | 37.3 |
| | (12.85) | (13.12) | (13.01) |
| Sex, n (%) | | | |
| Female | 114 | 111 | 225 |
| | (51.8) | (50.2) | (51.0) |
| Male | 106 | 110 | 216 |
| | (48.2) | (49.8) | (490) |
| BMI, kg/m², mean (SD) | 23.93 | 23.75 | 23.84 |
| | (4.78) | (4.52) | (4.65) |
| Time from diagnosis to first | 118.92 | 107.79 | 113.34 |
| study drug administration, | (109.66) | (101.24) | (105.55) |
| months, mean (SD) | | | |
| CDAI score, mean (SD) | 287.2 | 287.2 | 287.2 |
| | (55.00) | (64.74) | (60.02) |
| SES-CD score, mean (SD) | 13.4 | 12.7 | 13.1 |
| | (8.83) | (7.52) | (8.20) |
| CRP, mg/L, mean (SD) | 16.41 | 15.84 | 16.12 |
| | (23.82) | (23.38) | (23.58) |
| FCal, μg/g (SD) | 1,952.7 | 1,658.8 | 1,808.8 |
| | (3,496.13) | (2,466.32) | (3,035.54) |
| Prior exposure to biologics | | | |
| Biologic-naïve at baseline, n (%) | 85 | 84 | 169 |
| | (38.6) | (38.0) | (38.3) |
| Had prior exposure to 1 biologic for | 135 | 137 | 272 |
| treatment of Crohn's disease at | (61.4) | (62.0) | (61.7) |
| baseline, n (%)^{a} | | | |
| Location of disease, n, mean (5) | | | |
| N | 208 | 208 | 416 |
| Ileal | 54 | 54 | 108 |
| | (26.0) | (26.0) | (26.0) |
| Colonic | 72 | 83 | 155 |
| | (34.6) | (39.9) | (37.3) |
| Ileocolonic | 82 | 71 | 153 |
| | (39.4) | (34.1) | (36.8) |
| Patients with >1 concomitant | 215 | 213 | 428 |
| medication, n (%) | (97.7) | (96.4) | (97.1) |
| Systemic corticosteroids, | 37 | 33 | 70 |
| not including budesonide | (16.8) | (14.9) | (15.9) |
| Budesonide | 29 | 24 | 53 |
| | (13.2) | (10.9) | (12.0) |
| Immunosuppressants | 58 | 52 | 110 |
| | (26.4) | (23.5) | (24.9) |

TABLE 3
Safety Summary at Week 48

| AE, n (%) | T2T (n = 220) | SoC (n = 221) | RAS (N = 441) |
|---|---|---|---|
| Any AE | 189 (85.9) | 179 (81.0) | 368 (83.4) |
| AE related to study drug | 48 (21.8) | 54 (24.4) | 102 (23.1) |
| Any SAE | 27 (12.3) | 29 (13.1) | 56 (12.7) |
| SAE related to study drug | 4 (1.8) | 4 (1.8) | 8 (1.8) |
| AE leading to | 12 (5.5) | 20 (9.0) | 32 (7.3) |
| discontinuation | | | |
| AE leading to death | 2 (0.9) | 0 | 2 (0.5) |
| Common AE^{c} | | | |
| Infections and infestations | 102 (46.4) | 95 (43.0) | 197 (44.7) |
| Nasopharyngitis | 29 (13.2) | 29 (13.1) | 58 (13.2) |
| Gastrointestinal disorders | 88 (40.0) | 88 (39.8) | 176 (39.9) |
| Abdominal pain | 23 (10.5) | 19 (8.6) | 42 (9.5) |
| Musculoskeletal and | 48 (21.8) | 40 (18.1) | 88 (20.0) |
| connective tissue disorders | | | |
| Arthralgia | 24 (10.9) | 19 (8.6) | 43 (9.8) |
| Nervous system disorders | 35 (15.9) | 31 (14.0) | 66 (15.0) |
| Headache | 24 (10.9) | 21 (9.5) | 45 (10.2) |
| General disorders and | 49 (22.3) | 38 (17.2) | 87 (19.7) |
| administration site | | | |
| conditions | | | |
| Skin and subcutaneous | 33 (15.0) | 28 (12.7) | 61 (13.8) |
| tissue disorders | | | |
| Serious AE | 27 (12.3) | 29 (13.1) | 56 (12.7) |
| Infections and | 4 (1.8%) | 12 (5.4%) | 16 (3.6%) |
| infestations | | | |
| AE associated with | 4 (1.8%) | 5 (2.3%) | 9 (2.0%) |
| infusion | | | |
| Injection site reactions | 2 (0.9%) | 2 (0.9%) | 4 (0.9%) |

An AE is categorized as related if assessed by the investigator as possibly, probably, or very likely related to study agent. AEs leading to death are based on AE outcome of Fatal. Cause of death—unknown and cardiovascular (unconfirmed by autopsy)—both deaths were unrelated to study drug accordingly to investigators judgement. AEs reported by at least 5% of patients. AE associated with infusion refers to events that occurred within 1 hour after infusion.

TABLE 4
Clinic Outcomes through Week 104

| Clinical | | | Endoscopic | | |
|---|---|---|---|---|---|
| outcomes, | | | outcomes, mRAS | | |
| mRAS (n = 323) | W48 | W104 | (n = 323) | W48 | W104 |
| Proportions of | 92.6%, | 70.9%, (65.6; | Proportions of | 43.7%, | 39.3%, |
| patients in | (89.1; 95.2), | 75.8), n = 229 | patients in | (38.2; 49.3), | (34.0; 44.9), |
| clinical | n = 299 | | endoscopic | n = 141 | n = 127 |
| response^{a,b} %, | | | response^{a,d} %, | | |
| (95% CI), n | | | (95% CI), n | | |
| Proportions of | 83.9%, | 68.4%, (63.0; | Proportions of | 17.0%, | 14.6%, |
| patients in | (79.4; 87.7), | 73.5), n = 221 | patients in | (13.1; 21.6), | (10.9; 18.9), |
| clinical | n = 271 | | endoscopic | n = 55 | n = 47 |
| remission^{a,c} %, | | | remission^{a,e} %, | | |
| (95% CI), n | | | (95% CI), n | | |
| Variable, mRAS | W0 | W8^{g} | W16 | W48 | W104 |
| (n = ) | | | | | |
| CDAI score^{f}, mean | 283.0 (58.2), | 127.4 (84.6) | 96.6 (64.8) | 79.6 (66.5) | 76.3 (72.4) |
| (SD), n | n = 323 | n = 323 | n = 323 | n = 323 | n = 323 |
| SES-CD levels^{f}, | 12.4 (7.8) | N/A | 7.2 (6.1) | 6.8 (6.2) | 6.2 (5.8) |
| mean (SD), n | n = 323 | | n = 147 | n = 323 | n = 323 |

The mRAS includes all patients who entered into the LTE period (from W48 to W104).
^{a}Patients with missing data were analysed as non-responders or non-remitters (NRI).
^{b}Defined as a ≥ 100-point reduction from the baseline CDAI score, or a CDAI score <150.
^{c}Defined as CDAI total score of, 150 points.
^{d}Defined as a reduction from baseline in SES-CD of ≥50%.
^{e}Defined as SES-CD of ≤2.
^{f}Patients who had missing data at the designated analysis timepoint had their last value carried forward (LOCF).
^{g}Endoscopy was not performed for patients at W8.
CDAI, Crohn's Disease Activity Index;
LTE, long-term extension;
mRAS, modified Randomized Analysis Set;
N/A, not applicable;
NRI, non-responder imputation;
SES-CD, Simple Endoscopic Score for Crohn's Disease;
SD, standard deviation;
W, week.

What is claimed:

1. A method of treating Crohn's disease in a subject in need thereof, comprising:
performing endoscopy on the subject prior to treatment to measure baseline simple endoscopic score for Crohn's disease (SES-CD) and baseline CDAI;
administering to the subject a pharmaceutical composition comprising a clinically proven safe and clinically proven effective amount of an anti-IL-12/IL-23p40 antibody, wherein the antibody comprises a heavy chain variable region and a light chain variable region, the heavy chain variable region comprising: a complementarity determining region heavy chain 1 (CDRH1) amino acid sequence of SEQ ID NO:1; a CDRH2 amino acid sequence of SEQ ID NO:2; and a CDRH3 amino acid sequence of SEQ ID NO:3; and the light chain variable region comprising: a complementarity determining region light chain 1 (CDRL1) amino acid sequence of SEQ ID NO:4; a CDRL2 amino acid sequence of SEQ ID NO:5; and a CDRL3 amino acid sequence of SEQ ID NO:6 in an initial weight based IV dose of 6 mg of antibody per kg weight of the subject and a subcutaneous dose of 90 mg of antibody 8 weeks after administration of the initial dose;
measuring (i) Crohn's-associated biomarkers selected from C-reactive protein (CRP) and/or faecal calprotectin (FCal) levels, and/or (ii) clinical symptoms selected from a CDAI and SES-CD of the subject 16 weeks after administration of the initial dose;
administering (iii) 90 mg of antibody by subcutaneous dose 16 weeks after administration of the initial dose and every four weeks after the subcutaneous dose at 16 weeks to subjects measured to have CDAI <220, less than 70 point improvement from baseline CDAI, CRP ≤10 mg/L and/or FCal ≤250 ug/g, or (iv) 90 mg of antibody by subcutaneous dose 16 weeks after administration of the initial dose and every eight weeks after the subcutaneous dose at 16 weeks to subjects measured to have less than a 25% improvement in SES-CD score versus baseline SES-CD score; and
measuring (i) Crohn's-associated biomarkers selected from C-reactive protein (CRP) and/or faecal calprotectin (FCal) levels, and/or (ii) clinical symptoms selected from a CDAI and SES-CD of the subject 48 weeks and/or 104 weeks after administration of the initial dose.

2. The method of claim 1, wherein the measuring at 16 weeks after initial administration of the initial dose is done with an endoscopy.

3. The method of claim 1, wherein the measuring at 16 weeks after initial administration is performed by intestinal ultrasound.

4. The method of claim 1, wherein the subject treated achieves endoscopic improvement and of at least 50% reduction in SES-CD from baseline SES-CD 48 weeks after the initial dose and/or 104 weeks after the initial dose.

5. The method of claim 1, wherein the subject treated achieves overall endoscopic remission (SES-CD score ≤2), mucosal healing, CDAI improvement of ≥70 from baseline CDAI, clinical response with ≥100 reduction from baseline CDAI score, CDAI total score of <150, and/or change from baseline of fCal and CRP.

6. The method of claim 1, wherein the antibody comprises a heavy chain variable region amino acid sequence of SEQ ID NO:7 and a light chain variable region amino acid sequence of SEQ ID NO:8.

7. The method of claim 1, wherein the antibody comprises a heavy chain amino acid sequence of SEQ ID NO:10 and a light chain amino acid sequence of SEQ ID NO:11.

8. The method of claim 1, wherein the subject has moderately to severely active Crohn's disease as measured by CDAI between 220-450 or simple endoscopic score SES-CD ≥3.

9. The method of claim 1, wherein the measuring at 48 weeks after initial administration is performed by endoscopy.

10. The method of claim 9, wherein the subject is in clinical remission at 48 weeks after initial administration.

11. The method of claim 9, wherein the subject is in clinical remission at 104 weeks after initial administration.

12. The method of claim 1, wherein the subject had previously failed or were intolerant of at least one therapy selected from the group consisting of an anti-TNF, vedolizumab, corticosteroids, azathioprine (AZA), and 6 mercaptopurine (6 MP), or the subject had demonstrated corticosteroid dependence.

13. The method of claim 1, wherein the pharmaceutical composition for intravenous administration further comprises a solution comprising 10 mM L-histidine, 8.5% (w/v) sucrose, 0.04% (w/v) polysorbate 80, 0.4 mg/mL L-methionine, and 20 μg/mL EDTA disodium salt, dehydrate, at pH 6.0.

14. The method of claim 1, wherein the pharmaceutical composition for subcutaneous administration further comprises a solution comprising 6.7 mM L-histidine, 7.6% (w/v) sucrose, 0.004% (w/v) polysorbate 80, at pH 6.0.
